(19) United States
(12) Patent Application Publication
(10) Pub. No.: US 2025/0143955 A1
(43) Pub. Date: May 8, 2025

(54) MASSAGE DEVICE FOR PROVIDING MASSAGE MODE FOR STRETCHING USER'S LEGS

(71) Applicant: BODYFRIEND CO., LTD., Seoul (KR)

(72) Inventors: Se Jong KIM, Seoul (KR); Jung Min HEO, Seoul (KR)

(21) Appl. No.: 18/836,895

(22) PCT Filed: Apr. 10, 2023
(86) PCT No.: PCT/KR2023/004803

§ 371 Date: Aug. 8, 2024

(30) Foreign Application Priority Data
Apr. 22, 2022 (KR) 10-2022-0050268
Jun. 3, 2022 (KR) 10-2022-0067897
Jul. 5, 2022 (KR) 10-2022-0082710
Jul. 15, 2022 (KR) 10-2022-0087300
Sep. 6, 2022 (KR) 10-2022-0112976
Dec. 1, 2022 (KR) 10-2022-0165557

(51) Int. Cl. A61H 9/00; A61M 21/00; A61M 21/02

(52) U.S. Cl. CPC A61H 9/0078; A61M 21/02; A61H 2201/0107; A61H 2201/0149; A61H 2201/5002; A61H 2201/5007; A61H 2201/5043; A61H 2205/06; A61H 2205/081; A61H 2205/086; A61H 2205/10; A61M 2021/0022; A61M 2021/0027

(57) ABSTRACT

A massage device includes a controller for controlling the massage device; a body massage unit including a massage module for massaging at least a part of the user's body under the control of the controller; and a leg massage unit for massaging the user's legs under the control of the controller. The leg massage unit includes a first leg massage unit for massaging one of the user's legs, and a second leg massage unit for massaging the user's other leg, with the first leg massage unit and the second leg massage unit able to operate independently.

### TECHNICAL FIELD

The present invention relates to a massage device that provides a massage mode for stretching user's legs, and more particularly, to a massage device that stretches user's legs through leg massage units capable of operating independently of each other.

### BACKGROUND ART

Massage is a medical supplementary therapy that controls the modulation of a subject's body by applying various types of mechanical stimulation to a part of the subject's body, such as kneading, pressing, pulling, tapping, or moving the part of the subject's body and improves blood circulation and relieves the subject's fatigue.

The increase in demand for massage has caused an increase in the demand for massage devices that provide artificial massage functions due to economic and time reasons. As the demand for relieving fatigue or stress by loosening tight muscles through massage increases, various massage devices that are efficient in terms of time and cost are being released.

Recently, as people's interest in their health has increased, the massage devices are changing from simply providing the massage functions to electronic devices that provide various additional functions and/or medical functions. Accordingly, research on a method of efficiently controlling a massage device is continuing.

In particular, since each user may have different areas of interest to massage and desired stimulation, research activities on a device and a method for providing an optimized type of massage to each user are increasing.

As an example, Korean Patent Laid-Open Publication No. 10-2020-0100561 discloses an example of a massage device in which a first leg massage unit that accommodates a right leg and a second leg massage unit that accommodates a left leg are divided, and the first leg massage unit and the second leg massage unit are driven independently of each other.

Meanwhile, in the case of the leg massage units driven independently of each other, there is a problem in that a part of the user's body gets caught between the leg massage units as the leg massage units are driven independently of each other. These problems may cause safety accidents.

Therefore, there is a need for research on a massage device capable of preventing safety accidents while including leg massage units driven independently of each other.

### DISCLOSURE

### Technical Problem

The present invention is directed to providing a massage device for providing a massage mode for stretching user's legs.

However, the problem to be solved by the present invention is not limited to the above-described problem, and includes problems that can be easily understood by those skilled in the art from this specification and the attached drawings.

### Technical Solution

One aspect of the present invention provides a massage device including: a controller that controls the massage device; a body massage unit that includes a massage module for massaging at least a part of a body of a user under control of the controller; and a leg massage unit that massages legs of the user under the control of the controller, in which the leg massage unit includes a first leg massage unit that massages one leg of the user, and a second leg massage unit that massages the other leg of the user, and the first leg massage unit and the second leg massage unit operate independently of each other, and the controller performs a leg stretching motion of stretching the one leg by increasing a length of the first leg massage unit in a state in which a length of the second leg massage unit is adjusted or maintained and adjusting an angle of the first leg massage unit in a state in which an angle of the second leg massage unit is maintained.

### Advantageous Effects

A massage device according to an embodiment of the present invention can extend and relax user's iliopsoas muscle by stretching user's legs.

The effects which can be achieved by the present disclosure are not limited to the above-described effects. That is, other objects that are not described may be obviously understood by those skilled in the art to which the present disclosure pertains from the following description.

### DESCRIPTION OF DRAWINGS

FIG. 1 is a diagram for describing a massage device according to an embodiment of the present invention.

FIG. 2 is a diagram for describing a main frame according to an embodiment of the present invention.

FIG. 3 is a diagram for describing a detailed configuration of the massage device according to the embodiment of the present invention.

FIG. 4 is a diagram illustrating an external device capable of communicating with the massage device according to the embodiment of the present invention.

FIG. 5 is a diagram for describing an individual rotation of leg massage units in the massage device according to the embodiment of the present invention.

FIG. 6 is a diagram for describing a principle of rotating the leg massage units of FIG. 5. Specifically, FIG. 6 is a diagram for describing the leg massage units and a leg angle adjustment module of FIG. 5.

FIG. 7 is a diagram illustrating a state in which the leg massage unit according to the embodiment of the present invention is connected to a leg connection frame.

FIG. 8 is a cross-sectional view taken along line A-A′ of FIG. 7.

FIG. 9 is a cross-sectional view taken along the line B-B′ of FIG. 7.

FIG. 10A is a diagram illustrating a state in which a shaft is connected to a center support bracket and a side support bracket according to an embodiment of the present invention, and FIG. 10B is a diagram illustrating a state in which a center rotation member rotates.

FIG. 11 is a schematic plan view of FIG. 10.

FIG. 12A is a cross-sectional view taken along line C-C′ of FIG. 10, and FIG. 12B is a cross-sectional view taken along line C-C′ of FIG. 10 according to another embodiment of the present invention.

FIG. 13A is a diagram illustrating a center support bracket according to another embodiment of the present invention, and FIG. 13B is a diagram illustrating a center support bracket according to still another embodiment of the present invention.
(a) and (b) of FIG. 14 are diagrams illustrating a process of fastening a center bushing to the center support bracket, and (c) and (d) of FIG. 14 are diagrams illustrating a process of fastening a side bushing to the side support bracket.

FIG. 15 is an enlarged view of part D of FIG. 7.

FIG. 16 is a diagram illustrating a state in which a safety sensor module according to an embodiment of the present invention is disassembled.

FIG. 17 is a diagram illustrating another embodiment of the safety sensor module according to an embodiment of the present invention.
(a) and (b) of FIG. 18 are diagrams illustrating a process of driving the safety sensor module according to an embodiment of the present invention.

FIG. 19 is a diagram schematically expressing a leg length adjustment module of a leg massage unit 2000.

FIG. 20 is a diagram for describing a principle of length adjustment of the leg massage unit 2000.

FIG. 21 is an enlarged view of part B of FIG. 19.

FIG. 22 is an enlarged view of part C of FIG. 19.

FIG. 23 is a diagram illustrating a foot massage unit according to an embodiment of the present invention.

FIG. 24 is a diagram illustrating an anti-jamming sensor in the leg massage unit according to the embodiment of the present invention.

FIG. 25 is a diagram illustrating a photo sensor in the leg massage unit according to the embodiment of the present invention.

FIG. 26 is an enlarged view of D of FIG. 25.

FIG. 27 is a diagram for describing a stretching mode and an exercise mode provided by the massage device according to an embodiment of the present invention.

FIG. 28 is a diagram for describing an example of the massage mode illustrated in FIG. 27.

FIG. 29 is a diagram illustrating an example of describing a reclining operation of a controller according to an embodiment of the present invention.

FIG. 30 is a flowchart for describing a piriformis muscle stretching mode illustrated in FIG. 27.

FIG. 31 is a diagram for describing the operation of the piriformis muscle stretching mode illustrated in FIG. 30.

FIG. 32 is a diagram for describing a body air cell and a leg air cell included in a body massage unit according to an embodiment of the present disclosure.

FIG. 33 is a flowchart for describing an iliopsoas muscle stretching mode illustrated in FIG. 27.

FIG. 34 is a diagram for describing each operation illustrated in FIG. 33.

FIG. 35 is a diagram for describing a first leg stretching motion illustrated in FIG. 33.

### BEST MODE

A massage device includes: a controller that controls the massage device; a body massage unit that includes a massage module for massaging at least a part of a body of a user under control of the controller; and a leg massage unit that massages legs of the user under the control of the controller.

The leg massage unit includes a first leg massage unit that massages one leg of the user, and a second leg massage unit that massages the other leg of the user, and the first leg massage unit and the second leg massage unit can operate independently of each other, and the controller performs a leg stretching motion of stretching the one leg by increasing a length of the first leg massage unit in a state in which a length of the second leg massage unit is adjusted or maintained and adjusting an angle of the first leg massage unit in a state in which an angle of the second leg massage unit is maintained.

### Modes of the Invention

Various advantages and features of disclosed embodiments and methods accomplishing them will become apparent from the following description of embodiments with reference to the accompanying drawings. However, the present disclosure is limited to exemplary embodiments disclosed below, but may be implemented in various different forms. These embodiments will be provided only in order to make the disclosure of the present invention complete and allow those skilled in the art to which the present disclosure pertains to completely recognize the scope of the present invention.

After terms used in the present specification are briefly described, the disclosed embodiments will be described in detail.

General terms that are currently widely used are selected as terms used in the present specification in consideration of functions in the present disclosure but may be changed depending on the intention of those skilled in the art or a judicial precedent, the emergence of a new technique, and the like. In addition, in a specific case, terms arbitrarily chosen by an applicant may exist. In this case, the meaning of such terms will be mentioned in detail in a corresponding description portion of the present invention. Therefore, the terms used in the present disclosure are to be defined on the basis of the meaning of the terms and the contents throughout the present invention rather than simple names of the terms.

In the present specification, singular forms include plural forms unless the context clearly indicates otherwise. In addition, plural forms include singular forms unless the context clearly indicates otherwise.

Throughout the specification, unless otherwise specified, “including” any component means that other components may be further included rather than excluding other components.

Also, as used herein, the term “unit” refers to a software or hardware component, and “unit” performs certain roles. However, the “unit” is not meant to be limited to software or hardware. The “unit” may be configured to be stored in a storage medium that can be addressed or may be configured to regenerate one or more processors. Accordingly, as an example, the “˜ unit” refers to components such as software components, object-oriented software components, class components, and task components, processes, functions, attributes, procedures, subroutines, segments of program code, drivers, firmware, microcode, circuits, data, databases, data structures, tables, arrays and variables. Components and functions provided within “unit” may be combined into a smaller number of components and “unit” or may be further separated into additional components and “unit”.

According to an embodiment of the present invention, the “unit” may be implemented as a processor and a memory. The term “processor” should be interpreted broadly to include a general purpose processor, a central processing unit (CPU), a microprocessor, a digital signal processor (DSP), a controller, a microcontroller, a state machine, and the like. In some contexts, the “processor” may refer to an application specific semiconductor (ASIC), a programmable logic device (PLD), a field programmable gate array (FPGA), or the like. The term “processor” may also refer to a combination of processing devices, such as a combination of a DSP and a microprocessor, a combination of a plurality of microprocessors, a combination of one or more microprocessors in combination with a DSP core, or combinations of any other such configurations.

The term “memory” should be interpreted broadly to include any electronic component capable of storing electronic information. The term memory may refer to various types of processor-readable media such as a random access memory (RAM), a read-only memory (ROM), a non-volatile random access memory (NVRAM), a programmable read-only memory (PROM), an erase-programmable read-only memory (EPROM), an electrically erasable PROM (EEPROM), a flash memory, magnetic or optical data storage, and registers. The memory is said to be in electronic communication with a processor when the processor is capable of reading and/or writing information from and/or to the memory. The memory integrated in the processor is in electronic communication with the processor.

In this specification, an actuator refers to a configuration that may provide driving force. For example, the actuator may include, but is not limited to, a motor, a linear motor, an electronic motor, a direct current (DC) motor, an alternating current (AC) motor, a linear actuator, an electric actuator, etc.

In this specification, according to an embodiment, a massage device may refer to a massage device including a massage unit that massages a part of a user's body. In this case, the massage unit may include at least one of a body massage unit, an arm massage unit, and a leg massage unit. In addition, according to another embodiment, the body massage unit, the arm massage unit, and the leg massage unit may exist as separate devices (e.g., a body massage device, an arm massage device, and a leg massage device), and the massage device may refer to at least one of a body massage device, an arm massage device, and a leg massage device.

Hereinafter, embodiments of the invention will be described in detail with reference to the accompanying drawings so that those skilled in the art to which the present invention pertains may easily practice the present invention. In the drawings, parts irrelevant to the description are omitted in order to clarify the description of the present invention.

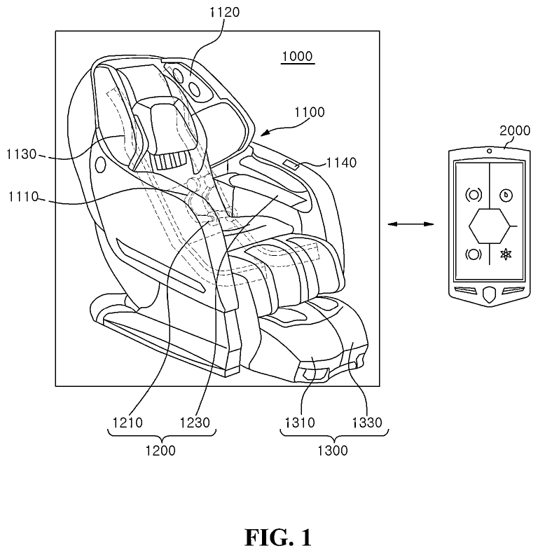

FIG. 1 is a diagram illustrating a massage device according to an embodiment of the present invention.

A massage device 1000 according to an embodiment of the present invention may include a massage unit that forms an area for accommodating at least a part of a user's body, supports the user's body (e.g., body), and massages a part of the user's body.

The massage unit may include at least one massage unit among a body massage unit 1100 that massages at least a part of the user's body, an arm massage unit 1200 that massages user's arms, and a leg massage unit 1300 that massages user's legs.

The body massage unit 1100 may massage at least a part of the user's body. For example, the body massage unit 1100 may massage at least a part of at least one of a user's upper body and lower body.

Specifically, the body massage unit 1100 may include a massage module 1110 that provides a massage function to at least a part of the user's body, an audio output module 1120 that provides any form of audio output to the user, a main frame 1130 that constitutes a framework of the body massage unit 1100 and supports the user's body, and a user input unit 1140 that receives any form of input from the user.

The components included in the body massage unit 1100 described above are merely exemplary embodiments, and the body massage unit 1100 may include various components in addition to the components described above.

In addition, the shape and structure of the massage device 1000 illustrated in FIG. 1 are merely exemplary, and various forms of massage devices 1000 may also be included within the scope of the present invention as long as they do not deviate from the scope defined by the claims of the present invention.

The body massage unit 1100 may form any shape of space for accommodating a user. The body massage unit 1100 may include a space having a shape corresponding to the shape of the user's body. For example, as illustrated in FIG. 1, the body massage unit 1100 may be implemented as a seated type that may accommodate the entire body of the user or a part of the body to support at least a part of the user's body. For example, the body massage unit 1100 may include a backrest part that supports at least a part of the user's upper body and a seat part that supports at least a part of the user's lower body.

A part in contact with the ground in the body massage unit 1100 may include any material to increase friction or any member (e.g., an anti-slip pad, etc.) to increase friction, and may include wheels to enhance mobility of the massage device 1000.

At least a part of the body massage unit 1100 may slide. For example, when the body massage unit 1100 starts massage (or when the body massage unit 1100 is driven), at least a part of the body massage unit 1100 may slide forward. In addition, the body massage unit 1100 may be tilted backward. As a result, the body massage unit 1100 may provide massage while tilted backward. For example, the massage device 1000 may include an actuator that tilts the massage device 1000 (or the body massage unit 1100) toward the rear of the massage device 1000 (or the body massage unit 1100). The massage device 1000 may drive the above-described actuator to tilt the body massage unit 1100 or restore the body massage unit 1100 to its original state. In this case, the actuator may be disposed in at least a part (e.g., base frame) of the massage device.

According to an embodiment of the present invention, the massage device 1000 may include at least one air cell (not illustrated). The air cell may be disposed in at least one of at least a partial area (e.g., at least one of an area corresponding to a user's shoulder, an area corresponding to a pelvis, and an area corresponding to a waist in the entire area of the body massage unit 1100) of the body massage unit 1100, at least a partial area of the arm massage unit 1200, and at least a partial area of the leg massage unit 1300, but is not limited thereto and may be disposed in various parts of the massage device 1000.

The massage device 1000 may include an air supply unit, and the air supply unit may inflate the air cell by supplying air to the air cell. The air supply unit may be located inside the body massage unit 1100 and may be located in the leg massage unit 1300. In addition, the air supply unit may be located outside the massage device 1000.

The massage module 1110 may be provided inside the body massage unit 1100 to provide any type of mechanical stimulation to the user accommodated in the body massage unit 1100. As illustrated in FIG. 1, the massage module 1110 may move along the main frame 1130 provided inside the body massage unit 1100. The massage module 1110 may massage at least a part of the user's body while moving along the main frame 1130.

The main frame 1130 of the body massage unit 1100 may be provided with a rack gear 1131a, and the massage module 1110 may provide mechanical stimulation to various parts of the user's body while moving along the rack gear. For example, the massage module 1110 may massage the user's body by tapping or massaging at least one of the user's back, shoulders, waist, hips, and thighs. The massage module 1110 may include at least one of a ball massage unit and a roller massage unit, but is not limited thereto.

The main frame 1130 constitutes a framework of the internal structure of the body massage unit 1100 and may be made of a metal material, a plastic material, etc. For example, the main frame 1130 may be made of iron, alloy, steel, etc., but is not limited thereto and may be made of various hard materials.

The audio output module 1120 may be provided in various locations. For example, the audio output module 1120 may include a plurality of output units such as a top audio output unit that is disposed on a top of the seat part in contact with the user, a front audio output unit that is attached to the front end of the arm massage units 1200 on left and right sides of the seat part, and/or a rear audio output unit attached to the rear end of the arm massage unit 1200, but is not limited thereto. In this case, the audio output module 1120 may provide three-dimensional sound such as a 5.1 channel, but is not limited thereto.

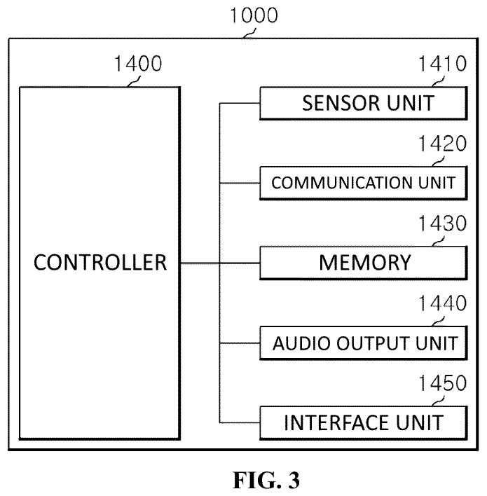

The arm massage unit 1200 may be disposed on at least one of one side and the other side of the body massage unit 1100. For example, the arm massage unit 1200 may include at least one of a first arm massage unit 1210 that is disposed on one side of the body massage unit 1100 to massage one arm of the user, and a second arm massage unit 1230 that is disposed on the other side of the body massage unit 1100 to massage the other arm of the user. The first arm massage unit 1210 may be disposed on one side of at least one of the seat part and the backrest part of the body massage unit 1100. The second arm massage unit 1230 may be disposed on the other side of at least one of the seat part and the backrest part of the body massage unit 1100. The first and second arm massage units 1210 and 1230 may operate independently of each other under control of a controller 1400 (see FIG. 3) that controls the massage device 1000.

The leg massage unit 1300 may be disposed below the body massage unit 1100 and/or below the front of the massage device 1000. The leg massage unit 1300 may include at least one of a first leg massage unit 1310 that is disposed on one side of the leg massage unit 1300 to massage one leg of the user, and a second leg massage unit 1330 that is disposed on the other side of the leg massage unit 1300 to massage the other leg of the user.

The leg massage unit 1300 includes an accommodation space for accommodating the user's legs, and may provide a leg massage to the user by massaging at least a part of the user's legs. For example, the leg massage unit 1300 may include a calf massage unit that massages a user's calf and/or a foot massage unit that massages user's feet.

The leg massage unit 1300 may adjust a length according to characteristics of the user's body. For example, when a tall user uses the massage device 1000, the leg massage unit 1300 needs to be lengthened due to the long length of the calf. In addition, when a short user uses the massage device 1000, the leg massage unit 1300 needs to be shortened due to the short length of the calf. Accordingly, the leg massage unit 1300 may provide a leg massage customized to a user's height. According to an embodiment of the present invention, the user may control the massage device 1000 using a massage device control device (CD). The massage device control device (CD) may be connected to the massage device 1000 through wired communication and/or wireless communication.

The massage device control device (CD) may include, but is not limited to, a personal device such as at least one of a remote controller, a cellular phone, and a personal digital assistant (PDA), and may include various electronic devices that can be connected to the massage device 1000 through the wired or wireless communication.

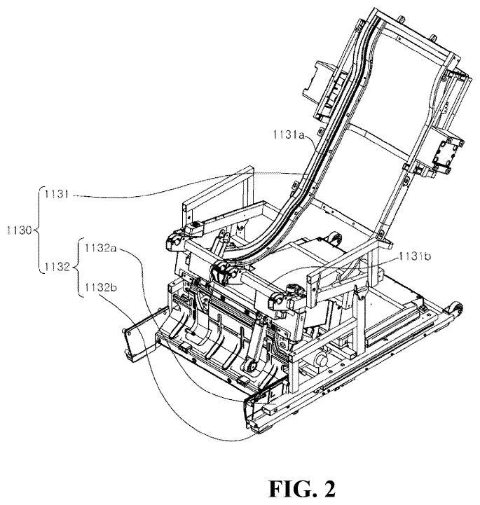

FIG. 2 is a diagram for describing the massage device according to the embodiment of the present invention.

According to an embodiment of the present invention, the main frame 1130 may include an upper frame 1131 on which the massage module 1110 is provided and a base frame 1132 that supports the upper frame 1131.

At least a part of the upper frame 1131 may be provided with a rack gear 1131a. The rack gear 1131a is a member for guiding vertical movement of the massage module 1110, and may include a plurality of valleys and a plurality of ridges.

According to an embodiment of the present invention, the rack gear 1131a may be provided in a form facing both side portions of the upper frame 1131, and the massage module 1110 may move along the rack gear 1131a.

In FIG. 2, the rack gear 1131a is formed in a vertical direction, but is not limited thereto. The rack gear 1131a may include a rack gear in the front-rear direction and a rack gear in the vertical direction. In the present disclosure, the front-rear direction may refer to a direction from the massage module 1110 toward the user or from the user toward the massage module 1110, and may refer to an X-axis direction based on FIG. 2.

The massage module 1110 may include a gear meshed with the rack gear 1131a. More specifically, the massage module 1110 may include gears meshed with the rack gear in the front-rear direction and the rack gear in the vertical direction, respectively. As the gear rotates by the actuator provided in the massage module 1110, the massage module 1110 may move forward, backward, upward, or downward.

As the massage module 1110 moves forward, the intensity of the massage may increase. In addition, as the massage module 1110 moves rearward, the intensity of the massage may become weaker.

The rack gear 1131a may be made of a metal material or a plastic material. For example, the rack gear 1131a may be implemented as iron, steel, alloy, reinforced plastic, melamine resin, phenolic resin, etc., but is not limited thereto.

The upper frame 1131 may be implemented in various shapes. For example, the upper frame 1131 may be divided into an S frame, an L frame, an S&L frame, and a double S&L frame depending on its shape, but is not limited thereto.

The S frame refers to a frame in which at least a part of the upper frame 1131 has a curved shape like an “S,” the L frame refers to a frame in which at least a part of the upper frame 1131 includes a curved shape like an “L,” the S&L frame refers to a frame that includes both a curved shape like an “S” and a curved shape like an “L,” and the double S&L frame refers to a frame that includes a curved shape like an “L” and a curved shape like a two-part “S”.

The base frame 1132 refers to a part that supports the upper frame 1131 and is in contact with the ground. The base frame 1132 may include a base upper frame 1132a and a base lower frame 1132b.

The base upper frame 1132a may support the upper frame 1131, and the base lower frame 1132b may be in contact with the ground. In addition, the base upper frame 1132a may be located in contact with the base lower frame 1132b.

According to an embodiment of the present invention, the base upper frame 1132a may move along the base lower frame 1132b. For example, the base upper frame 1132a may slide forward or backward along the base lower frame 1132b. In this case, the upper frame 1131 is connected to the base upper frame 1132a and may move according to the movement of the base upper frame 1132a.

For example, when the base upper frame 1132a moves forward, the upper frame 1131 may also move forward, and when the base upper frame 1132a moves backward, the upper frame 1131 may also move backward. As a result, the sliding movement of the body massage unit 1100 may be permitted.

To allow the movement of the base upper frame 1132a, a moving wheel may be provided on a lower portion of the base upper frame 1132a. In addition, a guide member capable of guiding the moving wheel may be provided on an upper portion of the base lower frame 1132b. The moving wheel provided on the base upper frame 1132a may move along the guide member provided on the base lower frame 1132b, thereby allowing the base upper frame 1132a to move forward or backward.

According to another embodiment of the present invention, the massage device 1000 may not provide a sliding function, and in this case, the base frame 1132 may not be separated into an upper and lower frame.

FIG. 3 is a diagram for describing the detailed configuration of the massage device according to an embodiment of the present invention.

According to an embodiment of the present invention, the massage device 1000 may include at least one of a controller 1400, a sensor unit 1410, a communication unit 1420, a memory 1430, an audio output unit 1440, and an interface unit 1450. The controller 1400 may include at least one processor and memory. At least one processor may execute instructions stored in the memory. For example, the processor may be the entity that performs the operation performed by the controller 1400. In other words, the processor may perform the operation of the controller 1400.

The controller 1400 may control the massage device 1000. For example, the controller 1400 may control at least one of the body massage unit 1100, the arm massage unit 1200, the leg massage unit 1300, the sensor unit 1410, the communication unit 1420, the memory 1430, the audio output unit 1440, the interface unit 1450, and the output unit. In addition, the controller 1400 may control various detailed configurations necessary for driving the massage device 1000 that are not described above.

The controller 1400 may include one processor or may include a plurality of processors. When the controller 1400 includes a plurality of processors, at least some of the plurality of processors may be located physically spaced apart from each other. In addition, the massage device 1000 is not limited thereto and may be implemented in various ways.

According to an embodiment of the present invention, the controller 1400 may control the operation of the massage device 1000. For example, the massage device 1000 may include a plurality of actuators, and the controller 1400 may control the operation of the massage device 1000 by controlling the operation of the plurality of actuators.

For example, the massage device 1000 may include at least one of a driving unit that moves an armrest frame support, at least one actuator included in the massage module 1110, a back angle actuator, a leg angle adjustment module, a foot massage actuator, a leg length adjustment module, and a sliding actuator. In this case, the leg angle adjustment module may be a leg angle actuator, and the leg length adjustment module may be a leg length adjustment actuator.

The back angle actuator is an actuator that adjusts an angle of the part of the massage device 1000 where the user's back is contacted, and the back angle of the massage device 1000 may be adjusted by the operation of the back angle actuator. The leg angle adjustment module is an actuator that adjusts the angle of the leg massage unit 1300 of the massage device 1000, and the angle between the leg massage unit 1300 and the body massage unit 1100 may be adjusted by the operation of the leg angle adjustment module. The foot massage actuator refers to an actuator that operates the foot massage module included in the leg massage unit 1300. For example, the controller 1400 may provide the foot massage to the user using a foot massage actuator. The leg length adjustment module represents the actuator that adjusts the length of the leg massage unit 1300. For example, the controller 1400 may use the leg length adjustment module to adjust the length of the leg massage unit 1300 to suit the user, and as a result, the user may receive a massage customized to his or her body type.

At least one actuator included in the massage module 1110 may be various actuators such as a movement actuator, a forward/backward actuator, and a massage actuator.

The movement actuator is an actuator that allows the massage module 1110 to move up and down, and may move the massage module 1110 along the main frame 1130 in at least one direction of the up direction and the down direction of the massage device 1000. The massage module 1110 may move along the rack gear by the operation of the movement actuator. The forward and backward actuator may advance and retreat (or tilt) the massage module 1110 in at least one of front and rear directions of the massage module 1110. The massage module 1110 may be tilted in at least one of the front and rear directions of the massage module 1110 through the forward and backward actuator. The massage actuator may provide various massage actions to the user by moving the massage module 1110 in various ways.

The controller 1400 may control the operation of the massage device 1000 by controlling the detailed components of the massage device 1000.

The controller 1400 may provide various massage operations by operating at least one actuator included in the massage device 1000. For example, the controller 1400 may provide a tapping massage, a kneading massage, etc., by operating at least one actuator included in the massage module 1110, but is not limited thereto and may provide various massage operations.

The memory 1430 may be included in the controller 1400 or may be outside the controller 1400. The memory 1430 may store various information related to the massage device 1000. For example, the memory 1430 may include massage control information and information about the user, but is not limited thereto. The information about the user may be various information related to the user, such as personal authentication information and user's biometric information (e.g., age, height, weight, blood pressure, body composition, heart rate, electrocardiogram, etc.).

The memory 1430 may be implemented through a non-volatile storage medium that may continuously store arbitrary data. For example, the memory 1430 may include, but is not limited to, disks, an optical disk, and a magneto-optical storage device, as well as storage devices based on flash memory and/or battery-backup memory.

The memory 1430 is a main storage device directly accessed by a processor, such as random access memory (RAM) such as dynamic random access memory (DRAM) and static random access memory (SRAM), and may refer to, but is not limited to, a volatile storage device in which the stored information is instantly erased when a power supply is turned off, but is not limited thereto. This memory 1430 may operate by the controller 1400.

The massage device 1000 may include the sensor unit 1410. The sensor unit 1410 may acquire various types of information using at least one sensor. The sensor unit 1410 may be provided as a sensor that uses measurement means such as pressure, potential, and optics. For example, the sensor may include, but is not limited to, pressure sensors, infrared sensors, LED sensors, touch sensors, electrode sensors, air pressure measurement sensors, etc.

The sensor unit 1410 may include a biometric information acquisition sensor. The biometric information acquisition sensor may acquire at least one of fingerprint information, face information, voice information, iris information, weight information, blood pressure information, heart rate (or heartbeat) information, electrocardiogram information, and body composition information, but is limited thereto, and may include various types of biometric information. The massage device 1000 may provide customized massage based on information acquired through sensors.

The massage device 1000 may detect a contact area and/or contact location with the user through the sensors. In addition, the massage device 1000 may provide customized massage based on information acquired through sensors. The sensor unit 1410 may include a safety sensor module 1500. The safety sensor module 1500 may control the driving of the leg massage unit 1300 or transmit a warning to the user when it is pressed by the user's body or by another object. A detailed description of the safety sensor module 1500 will be described later.

The communication unit 1420 may receive a signal transmitted from an external device or transmit a signal for the massage device 1000 to an external device.

For example, the controller 1400 may process a signal received through the communication unit 1420 to obtain a result signal and transmit the result signal to the communication unit 1420. The communication unit 1420 may transmit (or output) the result signal to an external device.

The controller 1400 may acquire information (e.g., information about the massage module 1110, information about the arm massage unit 1200, etc.) about various components included in the massage device 1000, and transmit the acquired information to the communication unit 1420. The communication unit 1420 may transmit the information about the massage device 1000 to an external device.

The communication unit 1420 may communicate with a module inside the massage device 1000, an external massage device, and/or a user terminal through any type of network. The communication unit may include a wired/wireless connection module for network connection. As the wireless connection technology, for example, a wireless LAN (WLAN) (Wi-Fi) technology, a wireless broadband (Wibro) technology, a world interoperability for microwave access (Wimax) technology, a high speed downlink packet access (HSDPA) technology, or the like, may be used. As the wired connection technology, for example, digital subscriber line (XDSL), fibers to the home (FTTH), power line communication (PLC), etc., may be used. In addition, the network connection unit includes a short-distance communication module and may transmit and receive data to and from any device/terminal located in a short distance. For example, the short range communication technology may include a Bluetooth technology, a radio frequency identification (RFID) technology, an infrared data association (IrDA) technology, an ultra wideband (UWB) technology, a ZigBee technology, but is not limited thereto.

The massage device 1000 may further include the interface unit 1450.

The interface unit 1450 may be disposed on at least a part of the massage device 1000. For example, the interface unit 1450 may be disposed on the outside of the arm massage unit 1200.

The interface unit 1450 may include an input unit and/or an output unit. The input unit may receive the input of the user input from the user and transmit the received input to the controller 1400. The output unit may display the information about the massage device 1000. For example, the output unit may output various types of information such as at least one of processing results of the controller 1400, operation information of at least one of the body massage unit 1100, the arm massage unit 1200, and the leg massage unit 1300, and information about the massage mode being executed.

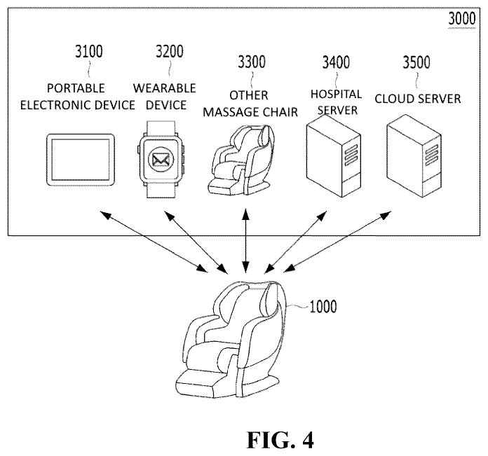

Specifically, the input unit may receive commands related to the operation control of the massage device 1000 from the user, and the input unit may be implemented in various forms. For example, the input unit may be provided in the body massage unit 1100 or the leg massage unit 1300, but is not limited thereto. In addition, the input unit may include the user input unit 1140 of FIG. 1, the massage device control device (CD), or various external devices that will be described in FIG. 4.

The massage device 1000 may acquire various commands from the user through the input unit. For example, the massage device 1000 may receive any command such as selection of a massage module, selection of a massage type, selection of massage intensity, selection of massage time, selection of a massage area, selection of the location and operation of the body massage unit 1100, selection of power on/off of the massage device 1000, selection of whether to operate a heating function, selection related to music playback, but is not limited thereto.

The massage device 1000 may provide an interface for selecting the massage mode through the interface unit 1450. For example, the input unit and/or output unit may include the massage device control device (CD). A list of various modes of medical massage related to body improvement may be listed through the massage device control device (CD).

The medical massage mode may include at least one of concentration mode, meditation mode, recovery mode, stretching mode, sleep mode, vitality mode, golf mode, hip-up mode, test taker mode, zero gravity mode, and growth mode.

According to another embodiment of the present invention, the interface unit 1450 may include buttons in the form of hot keys and/or selection buttons for executing a direction selection, cancellation, and input according to a preset user setting function, a self-preset function, or the like.

The interface unit 1450 may be implemented as a touch screen, a key pad, a dome switch, a touch pad (static pressure/electrostatic), a jog wheel, a jog switch, etc., but is not limited thereto. In addition, the interface unit 1450 may acquire commands through user's speech based on voice recognition technology.

The output unit may include a display for displaying the operation status of the massage device 1000, the current status of the user, etc. In this case, the display may include at least one of a liquid crystal display (LCD), a thin film transistor-liquid crystal display (TFT LCD), an organic light-emitting diode (OLED), a flexible display, and a 3D display, but is not limited thereto.

The output unit may include the audio output unit 1440. The audio output unit 1440 of FIG. 3 may include the audio output module 1120 of FIG. 1. For example, the audio output module 1120 may be disposed in various locations of the massage device 1000 as described above. As illustrated in FIG. 1, the audio output module 1120 may be disposed in an area corresponding to a user's head in the entire area of the body massage unit 1100 of the massage device 1000. In this case, the area corresponding to the user's head may be an area corresponding to at least one of the two upper side portions in the entire area of the body massage unit 1100.

The audio output unit 1440 may provide any type of audio output to the user. For example, the audio output unit 1440 may provide brain stimulation to the user by outputting sound sources and/or binaural beats optimized for the massage pattern provided by the massage device 1000 to the user. The audio output unit 1440 may output a sound signal received through a network (not illustrated) or stored in an internal/external storage medium (not illustrated). For example, the audio output unit 1440 may output a sound source under control of the user terminal through a network connection (e.g., Bluetooth connection, etc.) with the user terminal. In addition, the audio output unit 1440 may output any type of sound signal generated in connection with the operation of the massage device 1000.

Those skilled in the art will understand that the present invention can be implemented in combination with other program modules and/or as a combination of hardware and software. For example, the present invention may be implemented by computer-readable media.

Any medium that can be accessed by a computer can be a computer-readable medium, and such computer-readable media includes volatile and non-volatile media, transitory and non-transitory media, removable and non-removable media. As a non-limiting example, the computer-readable media may include computer-readable storage media and computer-readable transmission media.

The computer-readable storage media include volatile and non-volatile media, transient and non-transitory media, removable and non-removable media, implemented in any method or technology for storage of information such as computer-readable instructions, data structures, program modules, or other data. The computer-readable storage media may include RAM, ROM, EEPROM, flash memory or other memory technology, CD-ROM, digital video disk (DVD) or other optical disk storage, magnetic cassette, magnetic tape, magnetic disk storage or other magnetic storage, or any other medium that may be accessed by a computer and used to store desired information, but is not limited thereto.

FIG. 4 is a diagram illustrating an external device capable of communicating with the massage device 1000 according to an embodiment of the present invention.

The massage device 1000 may communicate wired or wirelessly with an external device 3000 through the communication unit 1420 to transmit and receive various data.

The external device 3000 may include a portable electronic device 3100 such as an AI speaker, tablet, or smartphone. The portable electronic device 3100 may be a dedicated massage device 1000 or may be a general-purpose portable electronic device. In addition, the external device 3000 may include a wearable device 3200 such as a smart watch or a smart band. The external device 3000 may include a massage device 3300 other than the massage device 1000 that the user is currently using. The external device 3000 may include a hospital server 3400. The external device 3000 may include a personal health record (PHR) server. In addition, the external device 3000 may include a cloud server 3500. The external device 3000 may include a medical measurement device such as an electronic scale, a blood sugar monitor, or a blood pressure monitor.

In the present invention, some examples of the external device 3000 are described, but any devices that may communicate with the massage device 1000 in a wired or wirelessly manner and exchange information with each other may be external devices.

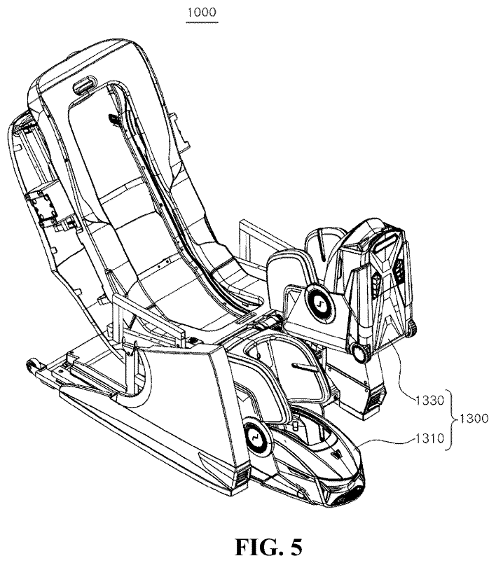

FIG. 5 is a diagram for describing an independent rotation of the leg massage unit in the massage device according to an embodiment of the present invention.

Referring to FIG. 5, the leg massage unit 1300 may be provided to correspond to both legs of the user. For example, the leg massage unit 1300 may include a first leg massage unit 1310 corresponding to one leg of the user and a second leg massage unit 1330 corresponding to the other leg of the user.

The first leg massage unit 1310 and the second leg massage unit 1330 may be controlled independently. For example, the first leg massage unit 1310 and the second leg massage unit 1330 may individually provide massage functions to the user. In addition, the first leg massage unit 1310 and the second leg massage unit 1330 may rotate independently. The user may receive different massages for both legs or provide alternate massages for both legs depending on need. In addition, the user may be provided with stretching or massage due to the rotation of both legs. In this case, the first leg massage unit 1310 and the second leg massage unit 1330 may rotate both legs of the user simultaneously or individually. In other words, the first leg massage unit 1310 and the second leg massage unit 1330 can operate independently of each other.

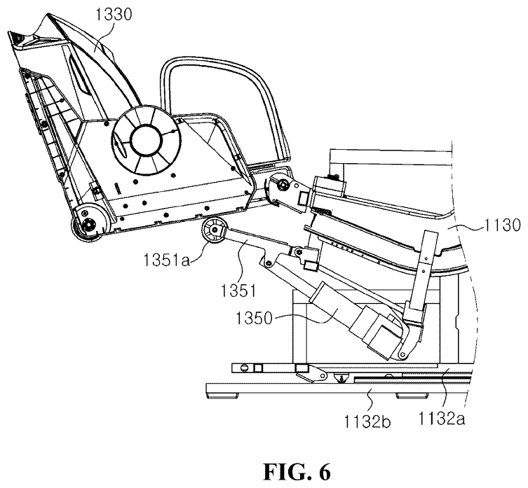

FIG. 6 is a diagram for describing the principle of rotation of the leg massage unit in FIG. 5. Specifically, FIG. 6 is a diagram for describing the leg massage unit and the leg angle adjustment module in FIG. 5.

Referring to FIG. 6, the massage device 1000 may include a leg angle adjustment module 1350 for rotating the leg massage unit 1300. The leg angle adjustment module 1350 may be disposed inside the body massage unit 1100. For example, the leg angle adjustment module may be connected to the main frame 1130.

The leg angle adjustment module 1350 may be driven to extend in one direction. As the leg angle adjustment module 1350 extends, the leg massage unit 1300 may rotate.

The leg angle adjustment module 1350 may include a link member 1351 at one end. The link member 1351 may contact the leg massage unit 1300 to provide rotational power to the leg massage unit 1300. A wheel 1351a may be provided at an end portion of the link member 1351 on the leg massage unit 1300 side.

The leg angle adjustment module 1350 may be provided to correspond to the first leg massage unit 1310 and the second leg massage unit 1330, respectively. The two leg angle adjustment modules 1350 may be connected to the first leg massage unit 1310 and the second leg massage unit 1330, respectively, to individually rotate the first leg massage unit 1310 and the second leg massage unit 1330.

For example, the leg angle adjustment module 1350 may include a first leg angle adjustment module 1350a and a second leg angle adjustment module 1350b.

The first leg angle adjustment module 1350a may be disposed in an area (or location) corresponding to the first leg massage unit 1310 in the entire area of the massage device 1000. For example, the first leg angle adjustment module 1350a may be disposed on one side of the lower portion of the body massage unit 1100 and/or behind the first leg massage unit 1310.

The first leg angle adjustment module 1350a may adjust the angle of the first leg massage unit 1310 by tilting (or rotating) the first leg massage unit 1310 in at least one of clockwise and counterclockwise directions. For example, the first leg angle adjustment module 1350a may be an actuator capable of changing the length of the first leg angle adjustment module 1350a. The first leg angle adjustment module 1350a may tilt the first leg massage unit 1310 by pushing or pulling the rear of the first leg massage unit 1310 by changing the length of the first leg angle adjustment module 1350a.

The second leg angle adjustment module 1350b may be disposed in an area (or location) corresponding to the second leg massage unit 1330 in the entire area of the massage device 1000. For example, the second leg angle adjustment module 1350b may be disposed on the other side of the lower portion of the body massage unit 1100 and/or behind the second leg massage unit 1330.

The second leg angle adjustment module 1350b may adjust the angle of the second leg massage unit 1330 by tilting the second leg massage unit 1330 in at least one of the clockwise and counterclockwise directions. For example, the second leg angle adjustment module 1350b may be an actuator capable of changing the length of the second leg angle adjustment module 1350b. The second leg angle adjustment module 1350b may tilt the second leg massage unit 1330 by changing the length of the second leg angle adjustment module 1350b to push or pull the rear of the second leg massage unit 1330.

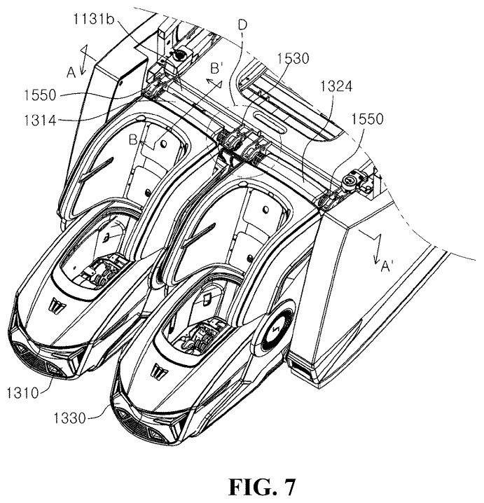

FIG. 7 is a diagram illustrating a state in which the leg massage unit according to an embodiment of the present invention is connected to a leg connection frame.

Referring to FIG. 7, the leg massage unit 1300 may be rotatably connected to the leg connection frame 1131b. For example, the leg connection frame 1131b may be disposed at one end (or lower end) of the body massage unit 1100 and may rotatably connect the first leg massage unit 1310 and the second leg massage unit 1330 to the leg connection frame 1131b.

The upper frame 1131 may include the leg connection frame 1131b. The leg connection frame 1131b may be provided at a front end of the upper frame 1131. The leg connection frame 1131b may be manufactured as a separate member and coupled to the upper frame 1131 or may be integrally manufactured with the upper frame 1131. When the leg connection frame 1131b is manufactured integrally with the upper frame 1131, the leg connection frame 1131b may mean one component of the upper frame 1131.

A center support bracket 1530 and a side support bracket 1550 may be connected to the leg connection frame 1131b. For example, the center support bracket 1530 and the side support bracket 1550 may be coupled to the leg connection frame 1131b by welding or fastening members such as bolts and rivets. However, the method of connecting the center support bracket 1530 and the side support bracket 1550 to the leg connection frame 1131b may be replaced by various methods commonly used in the technical field to which the present invention pertains.

The center support bracket 1530 and the side support bracket 1550 may rotatably fix the leg massage unit 1300.

The first leg massage unit 1310 and the second leg massage unit 1330 may be disposed in front of the leg connection frame 1131b. For example, the first leg massage unit 1310 may be disposed on the front right side of the leg connection frame 1131b, and the second leg massage unit 1330 may be disposed on the front left side of the leg connection frame 1131b.

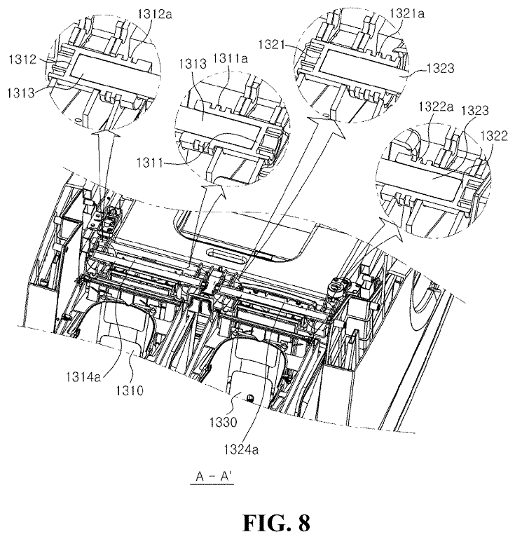

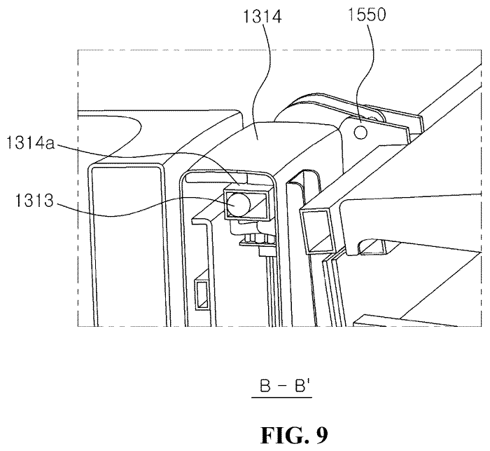

FIG. 8 is a cross-sectional view taken along line A-A′ of FIG. 7, and FIG. 9 is a cross-sectional view taken along line B-B′ of FIG. 7.

Referring to FIGS. 8 and 9, the first leg massage unit 1310 may include a first center bushing 1311, a first side bushing 1312, a first shaft 1313, and a first leg frame 1314. The first shaft 1313 may pass through the first leg frame 1314 and be coupled to the first leg frame 1314. For example, a first shaft housing 1314a may be provided inside the first leg frame 1314, and the first shaft 1313 may be coupled to the first shaft housing 1314a by passing therethrough. The first shaft 1313 may be fixed to the first shaft housing 1314a. For example, the first shaft 1313 may be coupled to the first shaft housing 1314a by welding or a separate fastening member. The method of coupling the first shaft 1313 to the first shaft housing 1314a is not limited to the above-described embodiment, and may be replaced with various methods commonly used in the technical field to which the present invention pertains.

Both end portions of the first shaft 1313 may be exposed to the outside of the first leg frame 1314. The first center bushing 1311 and the first side bushing 1312 may be coupled to both end portions of the first shaft 1313 exposed to the outside of the first leg frame 1314.

One end portion of the first shaft 1313 may be inserted and coupled to the first center bushing 1311, and the other end portion may be inserted and coupled to the first side bushing 1312.

The first center bushing 1311 and the first side bushing 1312 may include a resin. For example, the first center bushing 1311 and the first side bushing 1312 may include a synthetic resin. As an example, the synthetic resin may include polyacetal (POM). Since the first center bushing 1311 and the first side bushing 1312 include a synthetic resin, wear resistance and fatigue resistance may be improved.

In addition, the first center bushing 1311 and the first side bushing 1312 may include glass fiber. The corrosion resistance and insulation may be improved because the first center bushing 1311 and the first side bushing 1312 include the glass fiber.

It is also possible to integrally manufacture the first center bushing 1311, the first side bushing 1312, and the first shaft 1313. In this case, the first center bushing 1311, the first side bushing 1312, and the first shaft 1313 may be made of the same material. For example, the first center bushing 1311, the first side bushing 1312, and the first shaft 1313 may be made of metal material.

The first center bushing 1311 and the first side bushing 1312 may be rotatably fixed to the center support bracket 1530 and the side support bracket 1550, respectively.

A first center support rib 1311a may be provided to protrude from the outer peripheral surface of the first center bushing 1311 to prevent separation from the center support bracket 1530. The first center support rib 1311a may be provided to protrude outward in a radial direction. A bearing 1533 may be coupled to the outer peripheral surface of the first center bushing 1311 on which the first center support rib 1311a is not formed. The bearing 1533 may be supported by the center support bracket 1530.

A first side support rib 1312a may be provided to protrude from the outer peripheral surface of the first side bushing 1312 to prevent separation from the side support bracket 1550. The first side support rib 1312a may be provided to protrude outward in a radial direction. The bearing 1533 may be coupled to the outer peripheral surface of the first side bushing 1312 on which the first side support rib 1312a is not formed. The bearing 1533 may be supported by the side support bracket 1550.

The second leg massage unit 1330 may include a second center bushing 1321, a second side bushing 1322, a second shaft 1323, and a second leg frame 1324. A second center support rib 1321a may be provided on the outer peripheral surface of the second center bushing 1321, and the bearing 1533 may be coupled to the outer peripheral surface where the second center support rib 1321a is not formed. A second side support rib 1322a may be provided on the outer peripheral surface of the second side bushing 1322, and the bearing 1533 may be coupled to the outer peripheral surface where the second side support rib 1322a is not formed.

The above configuration is a component corresponding to each of the first center bushing 1311, the first side bushing 1312, the first shaft 1313, and the first leg frame 1314 of the first leg massage unit 1310, and the function and structure (left and right symmetry) thereof are the same. Therefore, a redundant description of the detailed configuration of the second leg massage unit 1330 will be omitted and replaced with the above description.

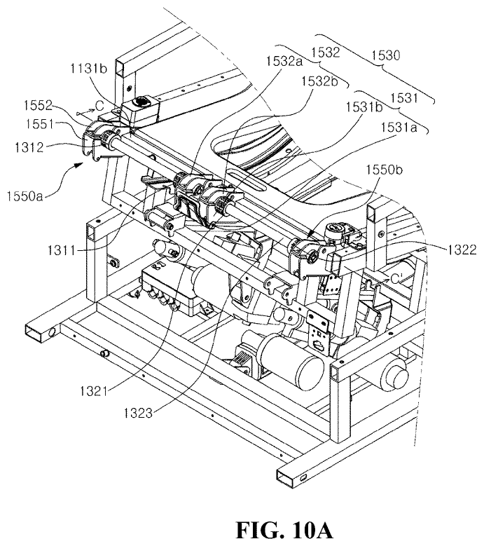

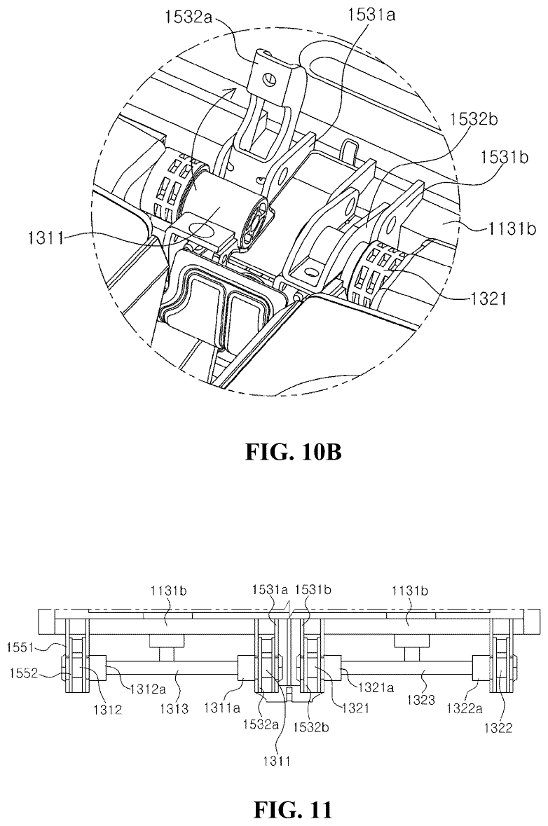

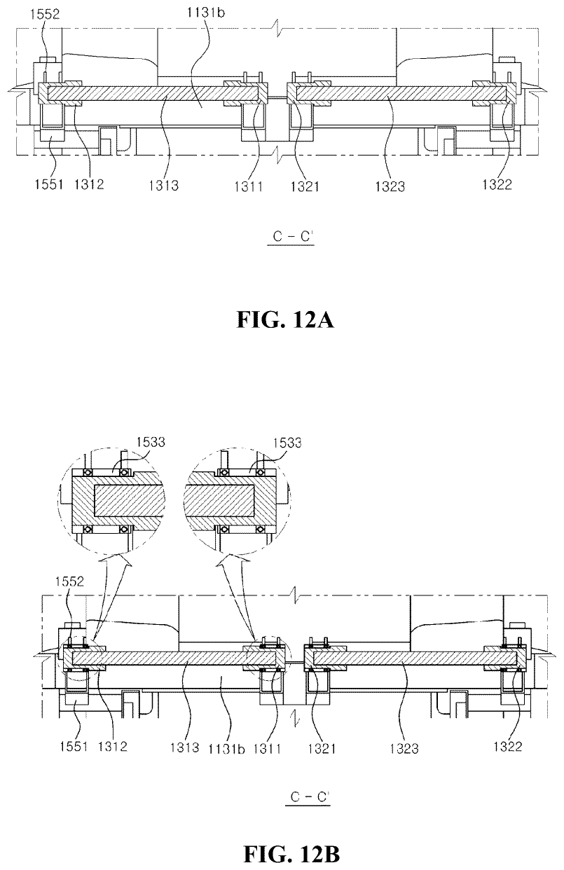

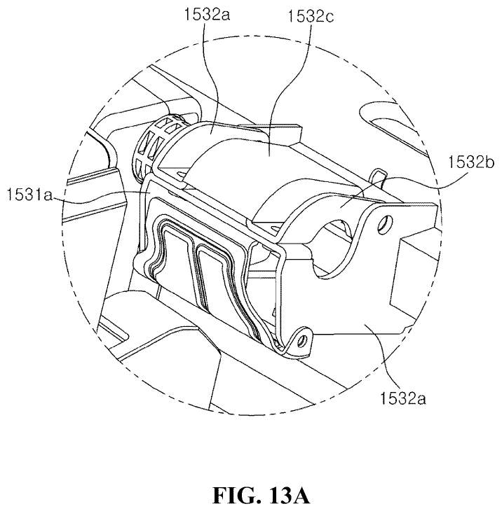

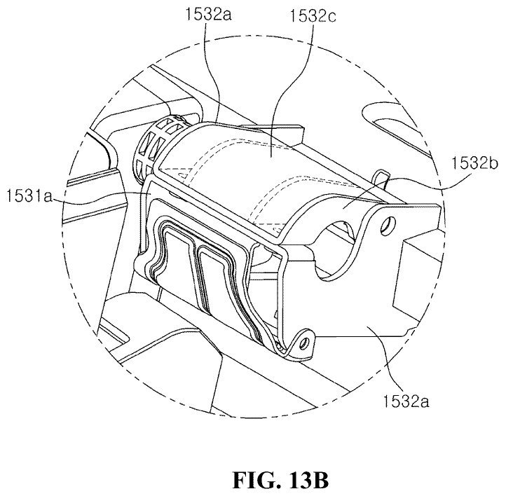

FIG. 10A is a diagram illustrating a state in which a shaft is connected to the center support bracket and the side support bracket according to an embodiment of the present invention, FIG. 10B is a diagram illustrating a state in which a center rotation member rotates, FIG. 11 is a schematic plan view of FIG. 10, FIG. 12A is a cross-sectional view taken along line C-C′ of FIG. 10, and FIG. 12B is a cross-sectional view taken along line C-C′ of FIG. 10 according to another embodiment of the present invention.

Referring to FIGS. 10 to 12, the center support bracket 1530 and the side support bracket 1550 may be coupled to the leg connection frame 1131b.

The center support bracket 1530 may be provided at a central portion of the leg connection frame 1131b. The center support bracket 1530 may rotatably fix the first center bushing 1311 of the first leg massage unit 1310 and the second center bushing 1321 of the second leg massage unit 1330.

Meanwhile, a bearing 1533 (see FIG. 12B) may be provided between the center support bracket 1530 and the center bushings 1311 and 1321 for smooth rotation. The bearing 1533 may be provided with various bearings used in the technical field to which the present invention pertains, such as ball bearings, thrust bearings, needle bearings, cylindrical bearings, and spherical roller bearings.

The center support bracket 1530 may include a center fixing member 1531 and a center rotation member 1532.

The center fixing member 1531 may be coupled to the leg connection frame 1131b by welding or a separate fastening member. One side of the center rotation member 1532 may be rotatably hinged to the center fixing member 1531. When the center rotation member 1532 rotates so that an end portion thereof comes into contact with the center fixing member 1531, the center rotation member 1532 may be fastened by the fastening member.

The center fixing member 1531 may include a first center fixing member 1531a and a second center fixing member 1531b. The first center fixing member 1531a and the second center fixing member 1531b may be provided in corresponding shapes.

The center rotation member 1532 may include a first center rotation member 1532a and a second center rotation member 1532b. The first center rotation member 1532a and the second center rotation member 1532b may be provided in corresponding shapes.

The first center rotation member 1532a and the second center rotation member 1532b may be rotatably coupled to the first center fixing member 1531a and the second center fixing member 1531b, respectively.

The first center fixing member 1531a and the first center rotation member 1532a may rotatably fix the first center bushing 1311. The second center fixing member 1531b and the second center rotation member 1532b may rotatably fix the second center bushing 1321.

FIG. 13A is a diagram illustrating a center support bracket according to another embodiment of the present invention, and FIG. 13B is a diagram illustrating a center support bracket according to still another embodiment of the present invention.

Referring to FIG. 13A, the first center rotation member 1532a and the second center rotation member 1532b may be connected by a center rotation member cover 1532c. For example, one side of the center rotation member cover 1532c may be connected to the inside of the first center rotation member 1532a, and the other side may be connected to the inside of the second center rotation member 1532b. The center rotation member cover 1532c may be coupled to the first center rotation member 1532a and the second center rotation member 1532b by welding or a separate fastening member. However, it is also possible to integrally manufacture the first center rotation member 1532a, the second center rotation member 1532b, and the center rotation member cover 1532c. In other words, the first center rotation member 1532a, the second center rotation member 1532b, and the center rotation member cover 1532c may be integrally provided by bending and cutting one member multiple times.

Referring to FIG. 13B, the center rotation member cover 1532c may be connected to the outside of the first center rotation member 1532a and the second center rotation member 1532b. The center rotation member cover 1532c may be coupled to the first center rotation member 1532a and the second center rotation member 1532b by welding or a separate fastening member. In this case, the center rotation member cover 1532c may be provided to entirely cover the upper portions of the first center rotation member 1532a and the second center rotation member 1532b.

In addition, it is also possible to connect the center rotation member cover 1532c to the first center rotation member 1532a and the second center rotation member 1532b through mechanical structures such as slide coupling or hook coupling. As long as the center rotation member cover 1532c is provided to cover at least a part of the upper portions of the first center rotation member 1532a and the second center rotation member 1532b, the configuration for coupling the center rotation member cover 1532c may be replaced by various methods commonly used in the technical field to which the present invention pertains.

The center support bracket 1530 may correspond to a component of the safety sensor module 1500, which will be described later. A detailed description of the detailed configuration of the center support bracket 1530 will be described later.

Side support brackets 1550 may be provided on both sides of the leg connection frame 1131b. The side support bracket 1550 may include a first side support bracket 1550a provided on the right end portion of the leg connection frame 1131b and a second side support bracket 1550b provided on the left end portion. The first side support bracket 1550a and the second side support bracket 1550b may be provided in corresponding shapes.

The first side support bracket 1550a may rotatably fix the first side bushing 1312. The second side support bracket 1550b may rotatably fix the second side bushing 1322. The bearings may be provided between the side support bracket 1550 and the side bushings 1312 and 1322 for smooth rotation.

The side support bracket 1550 may include, for example, a side fixing member 1551 and a side rotation member 1552.

The side fixing member 1551 may be coupled to the leg connection frame 1131b by welding or a separate fastening member. One side of the side rotation member 1552 may be rotatably coupled to the side fixing member 1551. When the side rotation member 1552 may rotate so that the end portion thereof comes into contact with the side fixing member 1551, the side rotation member 1552 may be fastened by the fastening member.

(a) and (b) of FIG. 14 are diagrams illustrating a process of fastening a center bushing to the center support bracket, and (c) and (d) of FIG. 14 are diagrams illustrating a process of fastening a side bushing to the side support bracket.

Referring to (a) and (b) of FIG. 14, the process of fastening the first center bushing 1311 to the center support bracket 1530 will be described.

The first center fixing member 1531a may be coupled to the leg connection frame 1131b. For example, the first center fixing member 1531a may be coupled to the leg connection frame 1131b by welding or a separate fastening member while the leg connection frame 1131b is inserted into a leg connection frame insertion groove 1531d-1 formed on one side of the first center fixing member 1531a.

When the first center fixing member 1531a is coupled to the leg connection frame 1131b, the first center rotation member 1532a may rotate upward. For example, the first center rotation member 1532a may be rotatably hinged to the first center fixing member 1531a through the center rotation axis 1532a-1, and may rotate upward based on the center rotation axis 1532a-1. Here, the center rotation axis 1532a-1 may mean a separate rotation axis rotatably connecting the first center fixing member 1531a and the first center rotation member 1532a. However, the center rotation axis 1532a-1 can be integrally provided with any one of the first center fixing member 1531a and the first center rotation member 1532a.

The first center bushing 1311 may be seated in a center bushing receiving groove 1531d-5 of the first center fixing member 1531a.

When the first center bushing 1311 is seated, the first center rotation member 1532a may rotate downward. In this case, at least a part of the outer peripheral surface of the upper portion of the first center bushing 1311 may be accommodated in a center bushing fixing groove 1532d of the first center rotation member 1532a. The center bushing receiving groove 1531d-5 and the center bushing fixing groove 1532d may rotatably support the first center bushing 1311.

When the first center rotation member 1532a rotates downward and the end portion contacts a fixing piece 1531d-4 of the first center fixing member, the first center rotation member 1532a may be coupled to the fixing piece 1531d-4 by the fastening member.

The process of fastening the second center bushing 1321 to the center support bracket 1530 is the same as the process of fastening the above-described first center bushing 1311 to the center support bracket 1530.

Referring to (b) and (c) of FIG. 14, the process of fastening the first side bushing 1312 to the first side support bracket 1550a will be described.

The side fixing member 1551 may be coupled to the leg connection frame 1131b. For example, the side fixing member 1551 may be coupled to the leg connection frame 1131b by welding or a separate fastening member.

When the side fixing member 1551 is coupled to the leg connection frame 1131b, the side rotation member 1552 may rotate upward. For example, the side rotation member 1552 may be rotatably hinged to the side fixing member 1551 through the side rotation axis 1552b, and may rotate upward based on the side rotation axis 1552b. Here, the side rotation axis 1552b may refer to a separate rotation axis rotatably connecting the side fixing member 1551 and the side rotation member 1552. However, the side rotation axis 1552b can be integrally provided with any one of the side fixing member 1551 and the side rotation member 1552.

The first side bushing 1312 may be seated in the side bushing receiving groove 1551a of the side fixing member 1551.

When the first side bushing 1312 is seated, the side rotation member 1552 may rotate downward. In this case, at least a part of the outer peripheral surface of the upper portion of the first side bushing 1312 may be accommodated in the side bushing fixing groove 1552a of the side rotation member 1552. The side bushing receiving groove 1551a and the side bushing fixing groove 1552a may rotatably support the first side bushing 1312.

When the side rotation member 1552 rotates downward and the end portion contacts the side fixing member 1551, the side rotation member 1552 may be fastened to the side fixing member 1551 by the fastening member.

The process of fastening the second side bushing 1322 to the second side support bracket 1550b is the same as the process of fastening the above-described first side bushing 1312 to the first side support bracket 1550a.

As described above, the center bushing and the side bushing may be fixed by being seated on and fastened to the center support bracket 1530 and the side support bracket 1550, so the leg massage unit 1300 may be easily connected to the body massage unit 1100.

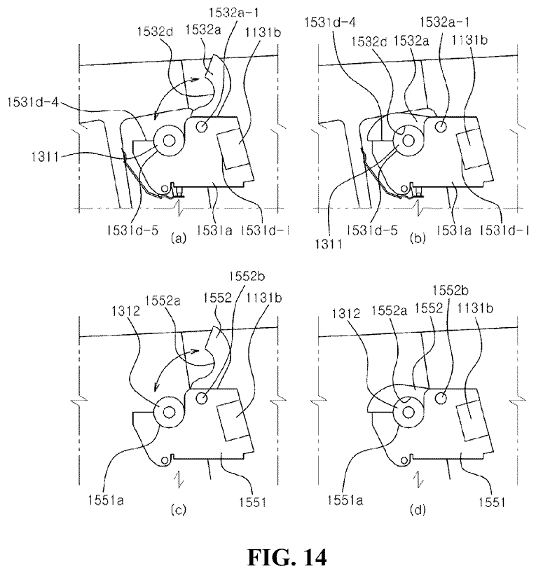

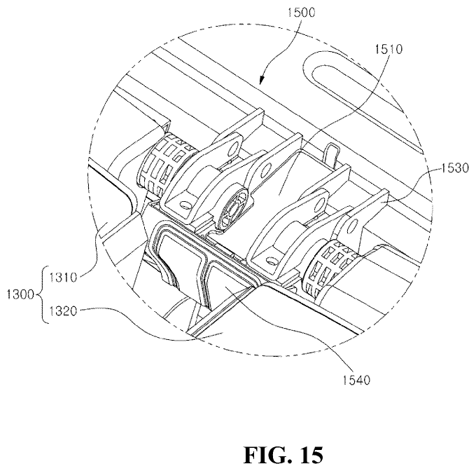

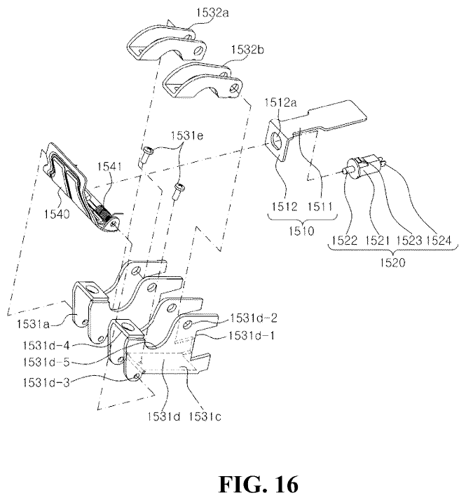

FIG. 15 is an enlarged view of part D of FIG. 7, FIG. 16 is a diagram illustrating a disassembled state of the safety sensor module according to an embodiment of the present invention, and FIG. 17 is a diagram illustrating another embodiment of the safety sensor module according to the embodiment of the present invention.

Referring to FIGS. 15 and 16, the massage device 1000 according to the embodiment of the present invention may include the safety sensor module 1500.

The safety sensor module 1500 may be provided in the leg connection frame 1131b. For example, the safety sensor module 1500 may be provided in a portion of the leg connection frame 1131b corresponding to a space between the first leg massage unit 1310 and the second leg massage unit 1330.

When applied with an external force, the safety sensor module 1500 may control the operation of at least one of the first leg massage unit 1310 and the second leg massage unit 1330. For example, when applied with the external force, the safety sensor module 1500 may stop the rotational operation of at least one of the first leg massage unit 1310 and the second leg massage unit 1330. In addition, when applied with the external force, the safety sensor module 1500 may rotate the first leg massage unit 1310 and the second leg massage unit 1330 at a predetermined angle in a direction opposite to the direction in which at least one of the first leg massage unit 1310 and the second leg massage unit 1330 rotates.

When applied with the external force, the safety sensor module 1500 may include an alarm unit (not illustrated) that transmits a warning signal to a user. The alarm unit may include, for example, at least one of a light emitting lamp, an audio, and a vibrating element. The light emitting lamp may transmit a warning signal by irradiating light to the user. The audio may correspond to the audio output module 1120 described above or may refer to a separate audio device. The audio may transmit the warning signal to the user through a warning sound. The vibrating element may be provided as a vibration panel, a vibration actuator, etc. The vibrating element is provided in at least one of the body massage unit 1100, the arm massage unit 1200, and the leg massage unit 1300 and may transmit the warning signal by applying vibration to the user. In this case, the light emitting lamp and/or the vibrating element may be disposed in the leg massage unit 1300. For example, the light emitting lamp may be disposed in various locations on the exterior (or outside) of the leg massage unit 1300. The vibrating element may be disposed in an area adjacent to the user's body (for example, an area corresponding to a sole, an area corresponding to a calf, etc.) in the entire area of the leg massage unit 1300.

Therefore, the safety sensor module 1500 may prevent safety accidents that may occur when the user's body becomes caught between the leg massage units 1300. In addition, the safety sensor module 1500 may prevent device damage/failure that may occur due to objects entering between the leg massage units 1300.

The safety sensor module 1500 may include a sensor bracket 1510, a sensor 1520, a center support bracket 1530, and a pressure plate 1540.

The sensor bracket 1510 may be provided to support the sensor 1520. The sensor bracket 1510 may be coupled to the leg connection frame 1131b. For example, the sensor bracket 1510 may be coupled to the leg connection frame 1131b by welding or a separate fastening member. The method of coupling the sensor bracket 1510 to the leg connection frame 1131b may be replaced by various methods commonly used in the technical field to which the present invention pertains.

The sensor bracket 1510 may include a protruding part 1511 and a bent part 1512. One side of the protruding part 1511 may be coupled to the leg connection frame 1131b, and the other side may protrude forward. The bent part 1512 may be bent downward at the protruding other end portion of the protruding part 1511. Accordingly, the sensor bracket 1510 may be provided in an overall ‘¬’ shape. The bent part 1512 may be provided with a sensor coupling hole 1512a.

The sensor 1520 may be coupled to the sensor bracket 1510. For example, the sensor 1520 may be coupled to the bent part 1512 of the sensor bracket 1510. In this case, at least a part of the sensor 1520 may be provided to penetrate through the sensor coupling hole 1512a. Accordingly, at least a part of the sensor 1520 may protrude in front of the bent part 1512.

The sensor 1520 may recognize the contact with the pressure plate 1540. For example, when an external force is applied to the pressure plate 1540 and the pressure plate 1540 rotates, the pressure plate 1540 may contact the sensor 1520. When the pressure plate 1540 is contacted, the sensor 1520 may transmit the corresponding information to the controller 1400. When the controller 1400 receives, from the sensor 1520, the information that the pressure plate 1540 has been contacted, as described above, the controller 1400 may control the operation of at least one of the first leg massage unit 1310 and the second leg massage unit 1330. In addition, the controller 1400 may drive the alarm unit to transmit the warning signal to the user.

The sensor 1520 may be provided as a sensor that uses measurement means such as pressure, potential, and optics. As an example, the sensor 1520 includes a sensor housing 1521, a contact part 1522 provided in the sensor housing 1521 and detecting the contact with the pressure plate 1540, a fastening part 1523 for fastening to the sensor bracket 1510, and an electrode terminal 1524 for electrical connection with an external device.

The contact part 1522 may slidably move inside the sensor housing 1521 when pressure is applied by the pressure plate 1540. For this purpose, a separate elastic member may be provided inside the sensor housing 1521 to apply elastic force to the contact part 1522. When the contact part 1522 is applied with pressure to slide to the inside of the sensor housing 1521, the sensor 1520 may transmit a signal to the controller 1400, and accordingly, as described above, the controller 1400 may control the driving of at least one of the first leg massage unit 1310 and the second leg massage unit 1330 or may transmit the warning signal to the user through the alarm unit.

The fastening part 1523 may be provided for coupling with the sensor bracket 1510. The fastening part 1523 may be made of a material with elastic force, and may be inserted into the sensor coupling hole 1512a formed in the sensor bracket 1510 when fastening the sensor 1520. In order to be easily inserted into the sensor coupling hole 1512a, the fastening part 1523 may include an inclined surface. The inclined surface may be provided so that the distance from the sensor housing 1521 increases as the inclined surface moves from the rear to the front of the sensor 1520 while one end portion is fixed to the sensor housing 1521. Additionally, the other end portion of the inclined surface may be provided with a flat surface for easy contact with the sensor coupling hole 1521a. The flat surface may be in contact with an inner side surface of the sensor coupling hole 1521a. The fastening part 1523 may be folded toward the sensor housing 1521 when held by the user. Therefore, when the sensor 1520 needs to be separated, the user may separate the sensor 1520 from the sensor bracket 1510 by holding the fastening part 1523.

Meanwhile, the sensor 1520 may be replaced with various sensors commonly used in the technical field to which the present invention pertains as long as it may recognize the rotation or contact of the pressure plate 1540. For example, the sensor 1520 may be provided as at least one of a pressure sensor, an infrared sensor, an LED sensor, a touch sensor, and an electrode sensor.

The center support bracket 1530 may be disposed on both sides of the sensor bracket 1510. The center support bracket 1530 may be manufactured as a separate member from the sensor bracket 1510 or may be manufactured integrally with the sensor bracket 1510. Alternatively, it is also possible to manufacture the center support bracket 1530 and the sensor bracket 1510 as separate members and then couple the center support bracket 1530 and the sensor bracket 1510 using a method such as welding. The method of coupling the center support bracket 1530 and the sensor bracket 1510 may be replaced by various methods commonly used in the technical field to which the present invention pertains. The pressure plate 1540 may be rotatably connected to the center support bracket 1530. The center bracket 1510 may include the center fixing member 1531 and the center rotation member 1532.

For example, the center fixing member 1531 may include a base part 1531c and side parts 1531d protruding upward from both sides of the base part 1531c. The base part 1531c and the side part 1531d may be formed by bending a single member. However, it is also possible to manufacture the base part 1531c and the side part 1531d as separate members and then couple the base part 1531c and the side part 1531d.

The base part 1531c may constitute a bottom surface of the center fixing member 1531. The base part 1531c may be provided with at least one stopper 1531e. The stopper 1531e may limit an angle at which the pressure plate 1540 rotates in a direction away from the sensor 1520. The stopper 1531e may penetrate through the base part 1531c so that an end portion protrudes downward from the base part 1531c. When the stopper 1531e is provided as a single stopper 1531c, the stopper 1531e may be disposed in a central area of the base part 1531c, and when the stopper 1531e is provided in plural numbers, the stoppers 1531e may be disposed in at least one of the central area and both areas of the base part 1531c.

The location and structure where the stopper 1531e is placed can be changed in various ways as long as the rotation angle of the pressure plate 1540 may be limited. For example, it is possible to attach the stopper 1531e to the bottom surface of the base part 1531c so that the entire stopper 1531c protrudes downward from the base part 1531c. In addition, it is also possible to integrally manufacture the stopper 1531e with the base part 1531c. In addition, it is also possible to provide a stopper 1531e on the pressure plate 1540.

The side part 1531d may include the leg connection frame insertion groove 1531d-1. The leg connection frame 1131b may be inserted into and coupled to the leg connection frame insertion groove 1531d-1. The leg connection frame insertion groove 1531d-1 may be provided by cutting one end portion of the side part 1531d opposite to the leg connection frame 1131b. The leg connection frame insertion groove 1531d-1 may be provided in a shape such that the inner side surface is joined to an outer surface of the leg connection frame 1131b. When the leg connection frame 1131b is inserted into the leg connection frame insertion groove 1531d-1, the portion where the side part 1531d and the leg connection frame 1131b contact may be welded. However, the method of coupling the side part 1531d and the leg connection frame 1131b may be replaced by the method commonly used in the technical field to which the present invention pertains.

The side part 1531d may include a center rotation member insertion hole 1531d-2. The center rotation member 1532 may be rotatably hinged to the center rotation member insertion hole 1531d-2. The center rotation member insertion hole 1531d-2 may be provided at various locations of the side part 1531d to allow the center rotation member 1532 to rotate at the upper portion of the side part 1531d.

The side part 1531d may include a pressure plate insertion hole 1531d-3. One side of the pressure plate 1540 may be rotatably hinged to the pressure plate insertion hole 1531d-3. When the other side of the pressure plate 1540 rotates upward (see FIG. 16) or rotates downward (see FIG. 17) and rotates at a preset angle or more, the pressure plate insertion hole 1531d-3 may be provided at various locations on the side part 1531d contacting the sensor 1520. For example, the pressure plate insertion hole 1531d-3 may be provided on the front lower side (see FIG. 16) or the front upper side (see FIG. 17) of the side part 1531d.

Any one of the side parts 1531d protruding from both sides of the base part 1531c may be provided with a fixing piece 1531d-4 bent toward the other side part 1531d. However, it is also possible to manufacture the fixing piece 1531d-4 as a separate member and couple the fixing piece 1531d-4 to both side parts 1531d. When the center rotation member 1532 rotates and the end portion contacts the fixing piece 1531d-4, the end portion of the center rotation member 1532 and the fixing piece 1531d-4 may be coupled by a fastening member.

The side part 1531d may include a center bushing receiving groove 1531d-5. The center bushings 1311 and 1321 may be rotatably seated in the center bushing receiving groove 1531d-5. For example, the center bushing receiving groove 1531d-5 may be provided by cutting the upper end portion of the side part 1531d.

The center rotation member 1532 may be rotatably hinged to the side part 1531d. The center rotation member 1532 may include the center bushing fixing groove 1532d. When the center rotation member 1532 rotates to be coupled to the fixing piece 1531d-4 of the side part 1531d, the center bushing fixing groove 1532d may rotatably support the center bushings 1311 and 1321 together with the center bushing receiving groove 1531d-5.

The pressure plate 1540 may be rotatably hinged to the center support bracket 1530. The pressure plate 1540 may include an elastic member 1541. The elastic member 1541 may apply an elastic force to the pressure plate 1540 so that a free end portion of the pressure plate 1540 rotates in a direction away from the sensor 1520. The elastic member 1541 may be provided as various members capable of applying the elastic force to the pressure plate 1540. As an example, the elastic member 1541 may be provided as a torsion spring. When the elastic member 1541 is provided as the torsion spring, when the pressure plate 1540 rotates, both end portions of the torsion spring may contact at least one of the pressure plate 1540 and the center support bracket 1530.

In a situation where the user's body or other objects are not caught between the leg massage units 1300 or between the leg massage unit 1300 and the body massage unit 1100, the clastic member 1541 may simply prevent the pressure plate 1540 from rotating due to the user's body or other objects coming into contact with the pressure plate 1540.

The pressure plate 1540 may rotate and come into contact with the sensor 1520. For example, when the external force greater than the elastic force of the elastic member 1541 is applied to the pressure plate 1540, the pressure plate 1540 may rotate and come into contact with the sensor 1520.

(a) and (b) of FIG. 18 are diagrams illustrating a process of driving the safety sensor module according to an embodiment of the present invention.

Referring to (a) of FIG. 18, one end portion of the pressure plate 1540 may be disposed to be spaced apart from the sensor 1520 by the elastic force of the clastic member 1541, and the other end portion may come into contact with the stopper 1531e. Since the elastic force is continuously applied to the pressure plate 1540, when a predetermined external force is not applied for a predetermined time, one end portion of the pressure plate 1540 may not come into contact with the sensor 1520.

Referring to (b) of FIG. 18, when at least one of the leg massage unit 1300 and the body massage unit 1100 rotates while the user's body is caught between the leg massage units 1300 and/or between the leg massage unit 1300 and the body massage unit 1100, the external force F greater than the elastic force of the elastic member 1541 may be applied to the pressure plate 1540. In addition, the external force F continues to be applied to the pressure plate 1540 unless the caught state is released. In this case, the pressure plate 1540 rotates, and one end portion may come into contact with the sensor 1520. When the pressure plate 1540 is contacted, the sensor 1520 may transmit the corresponding information to the controller 1400. When the controller 1400 receives the information from the sensor 1520 that the pressure plate 1540 has been contacted, the controller 1400 may control the driving of at least one of the first leg massage unit 1310, the second leg massage unit 1330, and the body massage unit 1100 or drive the alarm unit. Therefore, it is possible to prevent the safety accidents that may occur when the user's body is caught between the leg massage units 1300 and/or between the leg massage unit 1300 and the body massage unit 1100.

In addition, it is possible to prevent device failure and damage that may occur when objects other than the user's body are caught between the leg massage units 1300 or between the leg massage unit 1300 and the body massage unit 1100.

Hereinafter, for convenience of description, the leg massage unit 1300 changes to a leg massage unit 2000, the first leg massage unit 1310 changes to a first leg massage unit 2000a, and the second leg massage unit 1330 changes to a second leg massage unit 2000b.

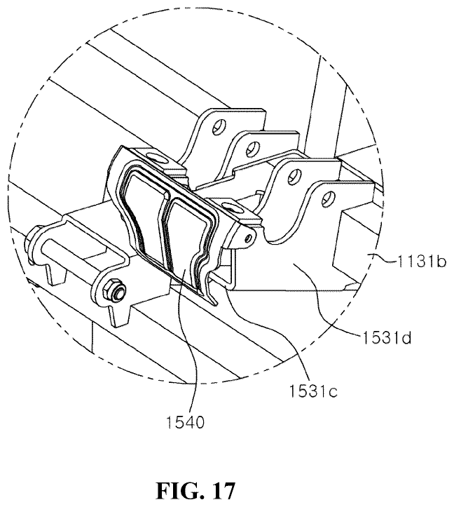

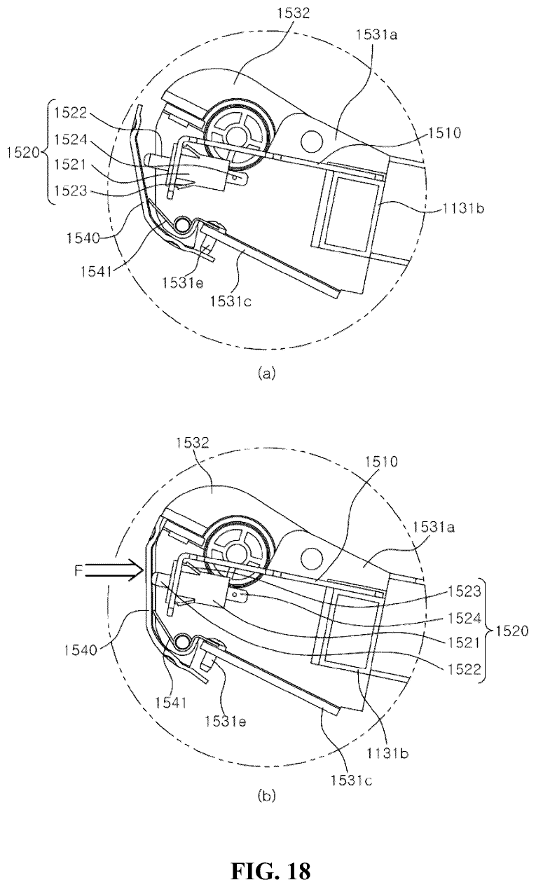

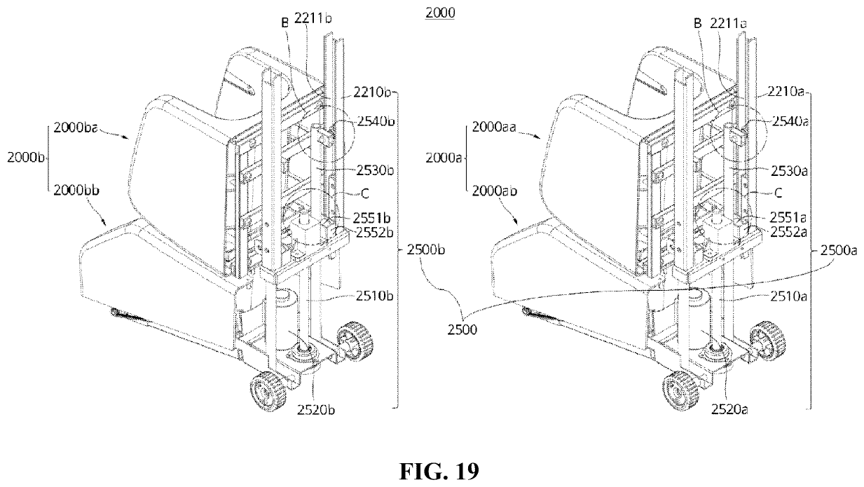

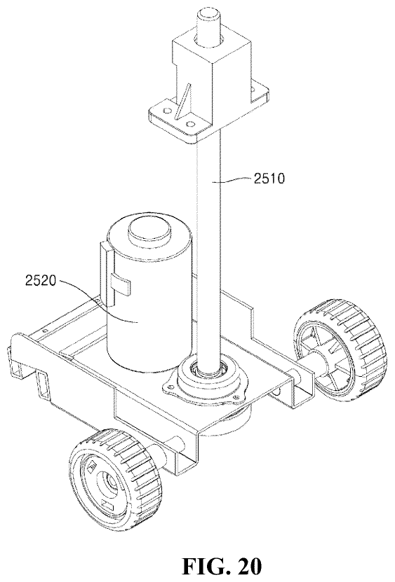

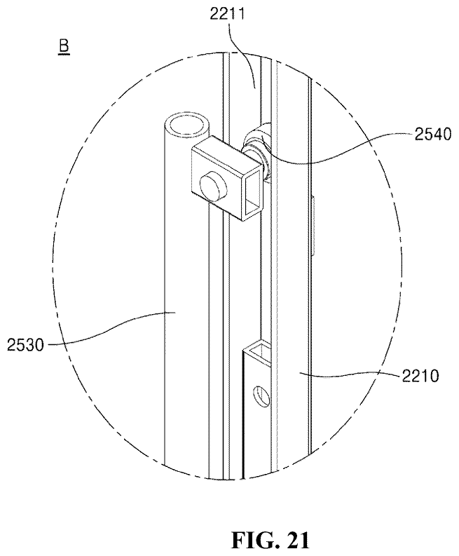

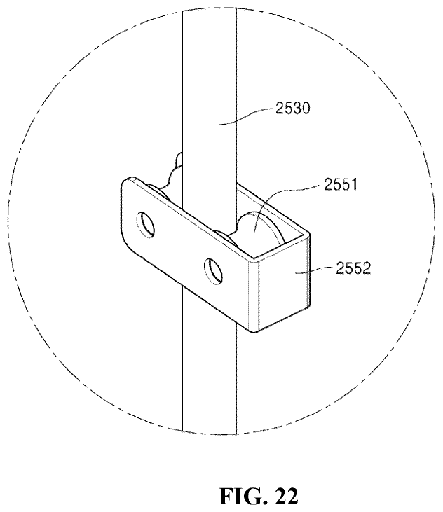

FIG. 19 is a diagram schematically illustrating a leg length adjustment module of the leg massage unit 2000 according to an embodiment of the present invention, and FIG. 20 is a diagram for describing a principle of length adjustment of the leg massage unit 2000 according to the embodiment of the present invention. FIG. 21 is an enlarged view of part B of FIG. 19, and FIG. 22 is an enlarged view of part C of FIG. 19.

Referring to FIG. 19, the massage device 1000 may include a leg length adjustment module 2500 that adjusts the length of the leg massage unit 2000. In this case, the leg length adjustment module 2500 may adjust the length of the leg massage unit 2000 under control of the controller 1400.

The leg length adjustment module 2500 may include a first leg length adjustment module 2500a and a second leg length adjustment module 2500b.

The first leg length adjustment module 2500a may be included in the first leg massage unit 2000a. For example, the first leg length adjustment module 2500a may be disposed behind the first leg massage unit 2000a. Specifically, the first leg length adjustment module 2500a may be disposed between a rear of the first leg massage unit 2000a and a front of the first leg angle adjustment module 1350a.

The first leg length adjustment module 2500a may adjust the length of the first leg massage unit 2000a by moving the location of the first leg massage unit 2000a. In this case, the first leg length adjustment module 2500a may adjust the length of the first leg massage unit 2000a based on a length of one leg of a user.

As an example, the first leg length adjustment module 2500a may control the length of the first leg massage unit 2000a by moving the first leg massage unit 2000a in at least one direction of the upper side (or up direction) and the lower side (or down direction) of the first leg massage unit 2000a. In this case, the up direction of the first leg massage unit 2000a may be a direction from the lower side of the first leg massage unit 2000a toward the upper side of the first leg massage unit 2000a, and the down direction of the first leg massage unit 2000a may be a direction from the upper side of the first leg massage unit 2000a toward the lower side of the first leg massage unit 2000a.

As another example, the first leg length adjustment module 2500a may move at least one of a first upper leg massage unit 2000aa and a first lower leg massage unit 2000ab of the first leg massage unit 2000a in at least one direction in the upper and lower sides of the first leg massage unit 2000a to adjust (or regulate) the distance between the first upper and first lower leg massage units 2000aa and 2000ab, thereby adjusting the length of the first leg massage unit 2000a. In this case, the first upper leg massage unit 2000aa may massage the upper portion (e.g., calf) of one leg of the user. For example, the first upper leg massage unit 2000aa may include a massage module that massages the upper portion of the leg, such as air cells and rollers, in at least a part of the entire area of the first upper leg massage unit 2000aa. The second lower leg massage unit 2000ab may massage the lower portion of the other leg of the user (e.g., feet). For example, the second lower leg massage unit 2000ab may include a massage module that massages the lower portion of the leg, such as air cells and rollers, in at least a part of the entire area of the second lower leg massage unit 2000ab.

The second leg length adjustment module 2500b may be included in the second leg massage unit 2000b. For example, the second leg length adjustment module 2500b may be disposed behind the second leg massage unit 2000b. Specifically, the second leg length adjustment module 2500b may be disposed between a rear of the second leg massage unit 2000b and a front of the second leg angle adjustment module 1350b.

The second leg length adjustment module 2500b may adjust the length of the second leg massage unit 2000b by moving the location of the second leg massage unit 2000b. In this case, the second leg length adjustment module 2500b may adjust the length of the second leg massage unit 2000b based on the length of the other leg of the user.

As an example, the second leg length adjustment module 2500b may control the length of the second leg massage unit 2000b by moving the second leg massage unit 2000b in at least one direction of the upper side (or up direction) and the lower side (or down direction) of the second leg massage unit 2000b. In this case, the up direction of the second leg massage unit 2000b may be a direction from the lower side of the second leg massage unit 2000b toward the upper side of the second leg massage unit 2000b, and the down direction of the second leg massage unit 2000b may be a direction from the upper side of the second leg massage unit 2000b toward the lower side of the second leg massage unit 2000b.

As another example, the second leg length adjustment module 2500b may move at least one of a second upper leg massage unit 2000ba and a second lower leg massage unit 2000bb of the second leg massage unit 2000b in at least one direction in the upper and lower sides of the second leg massage unit 2000b to adjust (or control) the distance between the second upper and second lower leg massage units 2000ba and 2000bb, thereby adjusting the length of the second leg massage unit 2000b. In this case, the contents of the first upper leg massage unit 2000aa described above may be applied to the second upper leg massage unit 2000ba, and the contents of the first lower leg massage unit 2000ab described above may be applied to the second lower leg massage unit 2000bb.

In other words, respective leg massage units 2000a and 2000b may adjust the lengths of respective leg massage units 2000a and 2000b based on the lengths of respective legs through the adjustment module of each leg massage unit.

Specifically, referring to FIGS. 19 and 20, the adjustment modules 2500a and 2500b may include lead screws 2510a and 2510b that adjust the distance between the upper leg massage units 2000aa and 2000ba and the lower leg massage units 2000ab and 2000bb and leg length adjustment actuators 2520a and 2520b that provide power to the lead screws 2510a and 2510b.

According to a preferred embodiment, the lead screws 2510a and 2510b may have a spiral formed along the outer surface, and the leg frame 2210 has an inner surface formed to correspond to the lead screws 2510a and 2510b and may include a fixing member through which the lead screws 2510a and 2510b pass. In addition, the leg frame 2210 may be provided in the upper leg massage units 2000aa and 2000ba, and the lead screws 2510a and 2510b and the leg length adjustment actuators 2520a and 2520b may be provided in the lower leg massage units 2000ab and 2000bb.

The lead screws 2510a and 2510b may rotate by the leg length adjustment actuators 2520a and 2520b, and as the lead screws 2510a and 2510b rotate, the lead screws 2510a and 2510b may move up and down while passing through the fixing member in a screw manner. As a result, the lower leg massage units 2000ab and 2000bb may move closer or further away from the upper leg massage units 2000aa and 2000ba as the lead screws 2510a and 2510b rotate, so the distance between the upper leg massage units 2000aa and 2000ba and the lower leg massage units 2000ab and 2000bb may be adjusted, thereby adjusting the lengths of the first and second leg massage units 2000a and 2000b.

Referring to FIG. 21, guide rails 2211a and 2211b may be formed inside the leg frames 2210a and 2210b. In addition, the first and second leg massage units 2000a and 2000b may include guide rollers 2540a and 2540b moving along guide rails 2211a and 2211b and guide bars 2530a and 2530b supporting the guide rollers 2540a and 2540b. In this case, one end of the guide bars 2530a and 2530b may be connected to the lower leg massage units 2000ab and 2000bb. Accordingly, as the lead screws 2510a and 2510b rotate, the guide bars 2530a and 2530b and the guide rollers 2540a and 2540b may move in the same direction as the lower leg massage units 2000ab and 2000bb.

Meanwhile, the guide rollers 2540a and 2540b may move along the guide rails 2211a and 2211b. Accordingly, the guide rollers 2540a and 2540b may guide a path to allow the lower leg massage units 2000ab and 2000bb to move in an appropriate direction. Accordingly, while the lower leg massage units 2000ab and 2000bb move, the leg massage units 2000a and 2000b on both sides may not shake.

Meanwhile, the guide rails 2211a and 2211b may be formed on a portion of an inside of the leg frame 2210. Referring to FIG. 21, the guide rails 2211a and 2211b are formed only on a portion of the inside of the leg frame 2210, and a jaw may be formed on the remaining portion to block the movement of the guide rollers 2540a and 2540b. As a result, it is possible to block the lower leg massage units 2000ab and 2000bb from moving beyond the limit point, and prevent the problem of the lower leg massage units 2000ab and 2000bb from completely separating from the upper leg massage units 2000aa and 2000ba.

Meanwhile, in order to more appropriately guide the movement of the guide bars 2530a and 2530b, the leg massage unit 2000 may further include guide wheels 2551a and 2551b and guide wheel brackets 2552a and 2552b that accommodate and support the guide wheels 2551a and 2551b. Referring to FIG. 22, at least one guide wheel 2551a and 2551b may be disposed inside the guide wheel brackets 2552a and 2552b. The outer surfaces of the guide wheels 2551a and 2551b may be depressed inward, and the guide bars 2530a and 2530b may be inserted and located to correspond to the depressed area. Accordingly, when the guide bars 2530a and 2530b move up and down, the guide wheels 2551a and 2551b may support the guide bars 2530a and 2530b so that they do not deviate from the moving path. As a result, the guide wheels 2551a and 2551b may support the guide bars 2530a and 2530b to prevent the guide bars 2530a and 2530b from shaking while moving, and guide a path to allow the guide bars 2530a and 2530b to move in an appropriate direction. Accordingly, the lower leg massage units 2000ab and 2000bb may also move in an appropriate direction and may not shake by the guide bars 2530a and 2530b.

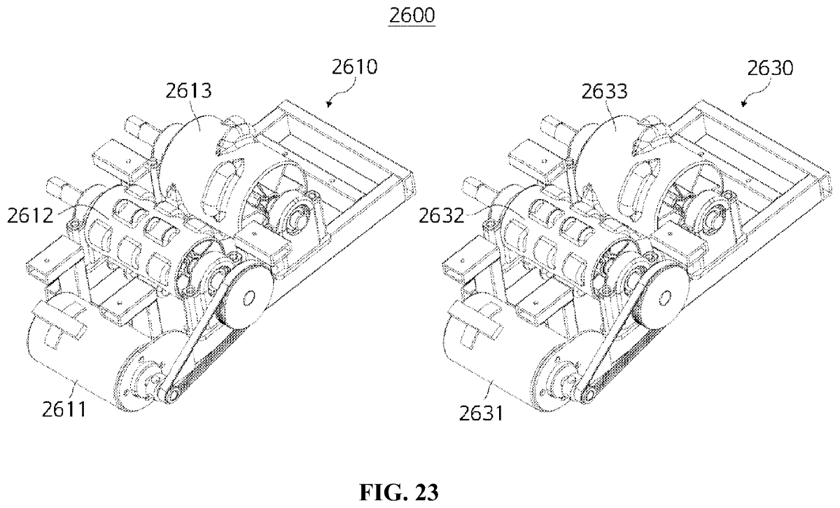

FIG. 23 is a diagram illustrating a foot massage unit 2600 according to an embodiment of the present invention.

The lower leg massage units 2000ab and 2000bb may include foot massage units 2610 and 2630 on one side. Preferably, the foot massage units 2610 and 2630 may be provided at the bottom or bottom surfaces of the lower leg massage units 2000ab and 2000bb.

The foot massage units 2610 and 2630 may include at least one foot massage module (or massage unit massaging feet) and a foot massage actuator (or foot driver) for driving at least one foot massage module. For example, the first leg massage unit 2000a may include a first foot massage unit 2610 disposed in an area corresponding to a sole of one leg of the user. The second leg massage unit 2000b may include a second foot massage unit 2630 disposed in an area corresponding to a sole of the other leg of the user.

The first foot massage unit 2610 may include at least one of the first foot massage module 2612 and the second foot massage module 2613. The first foot massage module 2612 and the second foot massage module 2613 are arranged (or disposed) at the front and rear of the first foot massage unit 2610, respectively, to massage the entire sole of one leg of the user. In addition, the first foot massage unit 2610 may include first foot massage actuators 2611 and 2613 that drive at least one foot massage module. The first foot massage actuators 2611 and 2613 may be disposed in front of the first foot massage unit 2610 and in front of the first foot massage module 2612 to drive at least one foot massage module. In this case, the front of the first foot massage unit 2610 and the front of the first foot massage module 2612 may be an area corresponding to toes of one leg in the entire area of the first foot massage unit 2610.

The second foot massage unit 2630 may include at least one of a third foot massage module 2632 and a fourth foot massage module 2633. For example, the third foot massage module 2632 and the fourth foot massage module 2633 may be arranged (or disposed) in front of and behind the second foot massage unit 2630, respectively, to massage the entire sole of the other leg of the user. In addition, the second foot massage unit 2630 may include the second foot massage actuator 2631 that drives at least one foot massage module. The second foot massage actuator 2631 may be disposed in front of the second foot massage unit 2630 and in front of the third foot massage module 2632 to drive at least one foot massage module. In this case, the front of the second foot massage unit 2630 and the front of the third foot massage module 2632 may be an area corresponding to toes of the other leg in the entire area of the second foot massage unit 2630.

As described above, the first and second foot massage units 2610 and 2630 include two foot massage modules and one foot massage actuator, but are not limited thereto. For example, each of the first and second foot massage units may include a plurality of foot massage modules (e.g., three or more), and each foot massage module may be disposed in a straight line as illustrated in FIG. 23. In this case, the straight line may be a line crossing the sole. Each of the first and second foot massage units may include a single foot massage actuator and drive all of the plurality of foot massage modules through the single foot massage actuator.

Meanwhile, the foot massage module may have at least one protrusion formed on its surface. Accordingly, the foot massage module may rotate to massage the user's sole.

Alternatively, the foot massage module may be a roller whose surface is formed in an arc shape. Accordingly, the foot massage module may rotate to massage the user's sole.

Meanwhile, as an optional embodiment, the foot massage actuator may be equipped with a cam. Therefore, the foot massage unit may provide acupressure massage to the user's toes using a cam without rolling massage. In addition, by mounting the cam on the foot massage actuator, it is possible to improve the spatial efficiency compared to the case where the foot massage actuator is provided separately.

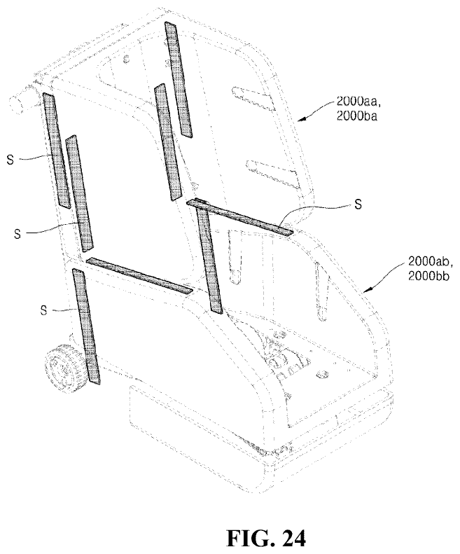

FIG. 24 is a diagram illustrating an anti-jamming sensor S in the leg massage unit 2000 according to the embodiment of the present invention.

At least one anti-jamming sensor S (or safety sensor) may be provided at at least one location between the side surface and rear surface of the leg massage unit 2000, and the upper leg massage units 2000aa and 2000ba and the lower leg massage units 2000ab and 2000bb. In this case, the anti-jamming sensor S may be a sensor for recognizing that a part of the user's body is caught around the leg massage units 2000a and 2000b.

In this case, the anti-jamming sensor S provided may be a capacitive touch sensor, but is not limited thereto.

In the case where the anti-jamming sensor S is attached to the side surface of the leg massage unit 2000, when the leg massage unit 2000 rotates, it may be recognized that a part of the user's body is caught between the leg massage units 2000a and 2000b on both sides. Alternatively, when the leg massage unit 2000 rotates, it may be recognized that a part of the user's body is caught between the leg massage unit 2000 and the body massage unit 1100.

In the case where the anti-jamming sensor S is attached to the rear surface of the leg massage unit 2000, when the leg massage unit 2000 rotates, it may be recognized that a part of the user's body is caught in the rear surface of the leg massage unit 2000.

In the case where the anti-jamming sensor S is attached between the upper leg massage units 2000aa and 2000ba and the lower leg massage units 2000ab and 2000bb, when the distances between the upper leg massage units 2000aa and 2000ba and the lower leg massage units 2000ab and 2000bb are adjusted, it may be recognized that a part of the user's body is caught between the upper leg massage units 2000aa and 2000ba and the lower leg massage units 2000ab and 2000bb.

Therefore, when the user's body is caught in a specific location of the massage device 1000 while the leg massage unit 2000 is rotating, the anti-jamming sensor S may recognize this, so the massage device 1000 may be stopped and the safety accidents may be prevented.

Meanwhile, the anti-jamming sensor S may be formed to be extended to have a length. That is, the anti-jamming sensor S may be a linear sensor.

When the leg massage unit 2000 rotates or the length is adjusted, it is impossible to know which part of the leg massage unit 2000 the human body will get stuck in, so when the anti-jamming sensor S is formed by extending in a longitudinal direction, the area that may detect whether the user's body is in contact may be expanded. Accordingly, the user may use the massage device 1000 more safely.

As an optional embodiment, the anti-jamming sensor S may be provided in the upper leg massage units 2000aa and 2000ba and the lower leg massage units 2000ab and 2000bb, respectively. At least one anti-jamming sensor S may be provided on each surface. For example, the anti-jamming sensor S may be provided on both side surfaces of the upper leg massage units 2000aa and 2000ba, respectively, and may be located on the left and right sides of the rear surface of the upper leg massage units 2000aa and 2000ba, respectively. In addition, the anti-jamming sensor S may be located on the surfaces between the upper leg massage units 2000aa and 2000ba and the lower leg massage units 2000ab and 2000bb. In addition, the anti-jamming sensor S may be provided on both side surfaces of the lower leg massage units 2000ab and 2000bb, respectively.

Therefore, even if a part of the user's body, such as the user's legs, gets caught somewhere adjacent to the leg massage unit 2000 while the massage device 1000 is operating, this situation may be recognized.

Although not illustrated in the drawing, the leg massage unit 2000 may further include a sensor for controlling the length of the leg massage unit 2000.

As an embodiment, the leg massage unit 2000 may include at least one limit sensor in the leg frame 2210. The limit sensor may be a sensor for determining the length adjustment limit of the leg massage unit 2000. Alternatively, the limit sensor may be a sensor for determining a reference location of the leg massage unit 2000. For example, the limit sensor may be a Hall sensor, but is not limited thereto.

By including the limit sensor, it is possible to keep the upper leg massage units 2000aa and 2000ba and the lower leg massage units 2000ab and 2000bb from being spaced apart from each other beyond a certain limit, prevent safety accidents, and prevent the lower leg massage units 2000ab and 2000bb from being completely separated from the upper leg massage units 2000aa and 2000ba.

As another embodiment, the leg massage unit 2000 may include an encoder. Therefore, the distances between the upper leg massage units 2000aa and 2000ba and the lower leg massage units 2000ab and 2000bb may be recognized using the encoder, and the length adjustment limit of the leg massage unit 2000 may be determined accordingly. In other words, the distances between the upper leg massage units 2000aa and 2000ba and the lower leg massage units 2000ab and 2000bb may be recognized through the encoder, and when a certain limit is reached, the relative movement of the upper leg massage units 2000aa and 2000ba and the lower leg massage units 2000ab and 2000bb may be stopped.

As an embodiment, the leg massage units 2000a and 2000b may further include a measurement sensor unit for measuring the lengths of the user's legs. For example, the measurement sensor unit may include at least one of a first measurement sensor and a second measurement sensor. The first leg massage unit 2000a may include the first measurement sensor that measures the length of one leg, and the second leg massage unit 2000b may include the second measurement sensor that measures the length of the other leg.

Preferably, each measurement sensor may include at least one (or sub-measurement sensor) of a first sensor (or first sub-measurement sensor) and a second sensor (or second sub-measurement sensor) to measure the lengths of the user's legs.

For example, the first sensor may be a capacitive touch sensor. The first sensor may be used to roughly measure the lengths of the user's legs. Accordingly, the lengths of the user's legs may be measured through the first sensor, and the lengths of the leg massage units 2000a and 2000b may be adjusted to correspond thereto.

The first sensor may recognize that the user's body is contacted to measure the lengths of the legs. Therefore, the first sensor may measure the lengths of the user's legs depending on whether the user's body is contacted, and the massage device 1000 may adjust the lengths of the leg massage units 2000a and 2000b based on the measurement, so the accuracy may be low, but the lengths of the leg massage units 2000a and 2000b may be adjusted quickly and roughly.

In order to precisely measure the lengths of the leg massage units 2000a and 2000b, the leg massage units 2000a and 2000b according to the present invention may further include the second sensor.

For example, the second sensor may be a photo sensor or a load sensor. The second sensor may be used to precisely measure the lengths of the user's legs. Therefore, the massage device 1000 may roughly measure the lengths of the user's legs using the first sensor, adjust the lengths of the leg massage units 2000a and 2000b to correspond thereto, and then precisely measure the lengths of the user's legs using the second sensor and adjust the lengths of the leg massage units 2000a and 2000b to correspond thereto.

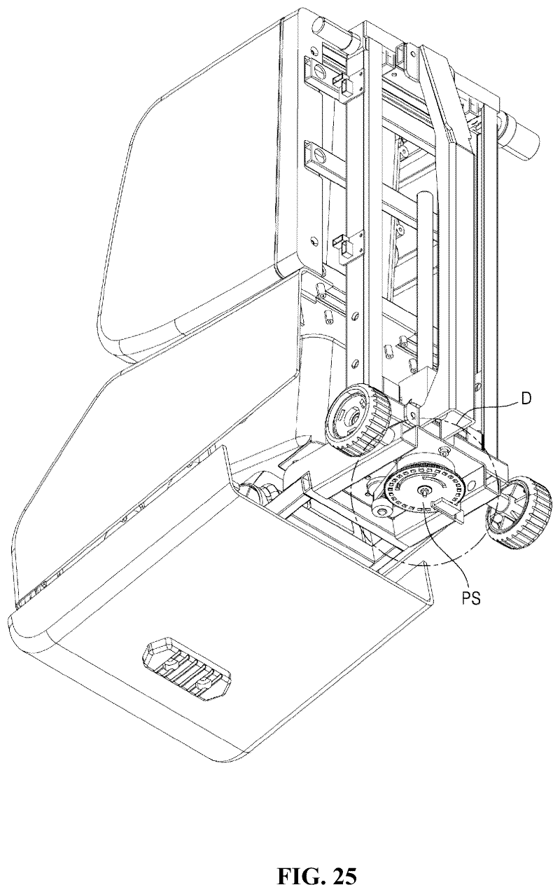

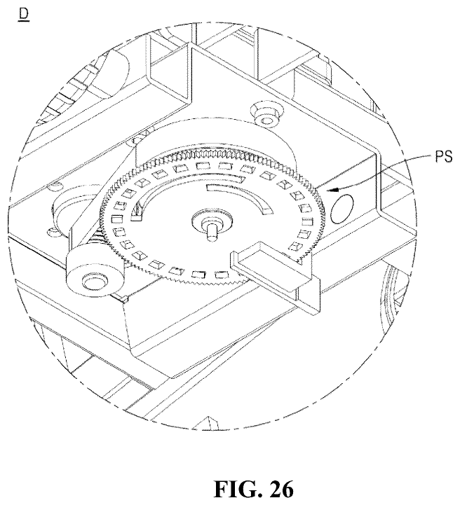

FIG. 25 is a diagram illustrating a photo sensor in a leg massage unit according to an embodiment of the present invention, and FIG. 26 is an enlarged view of part D of FIG. 25.

Referring to FIGS. 25 and 26, in an embodiment, the second sensor may be a photo sensor PS. Preferably, the second sensor may be a mechanical photo sensor. In this way, when using the photo sensor PS, as described above, the lengths of the user's legs may be measured more precisely compared to when only the first sensor is used. In addition, the photo sensors PS are provided in each leg massage unit on both sides, making it possible to precisely measure the lengths of each of the user's legs.

For example, the photo sensor PS may be located on the lower sides of the leg massage units 2000a and 2000b. Therefore, the photo sensor PS may measure the lengths of the user's legs more precisely. That is, referring to FIG. 26, the photo sensors PS are located on the lower sides of the leg massage units 2000a and 2000b on both sides, respectively, and when the movement of the leg massage units 2000a and 2000b occurs, a disk member rotates due to the movement, and by measuring the degree of light reception that changes due to the rotation of the disk member, it is possible to precisely measure the lengths of the user's legs.

However, it is not limited thereto, and of course, the lengths of the user's legs may be measured by using a different type of photo sensors PS or by using the principle of driving the existing photo sensor PS.

As another embodiment, the second sensor may measure a leg length by measuring the load applied by the user's legs.

For example, the controller 1400 of the massage device 1000 may recognize the lengths of the leg massage units 2000a and 2000b as the lengths of the user's legs when a preset size load is applied to the second sensor while adjusting the distance between the upper leg massage units 2000aa and 2000ba and the lower leg massage units 2000ab and 2000bb. Accordingly, the massage device 1000 may recognize the distance between the leg massage units 2000a and 2000b as the lengths of the user's legs when a preset load of a certain size is applied to the second sensor while finely adjusting the distances between the upper leg massage units 2000aa and 2000ba and the lower leg massage units 2000ab and 2000bb, and in this case, adjust the lengths of the leg massage units 2000a and 2000b to correspond to the lengths of the user's legs.

Meanwhile, the first sensor and the second sensor may be located on the lower surfaces of the leg massage units 2000a and 2000b. Preferably, the first sensor and the second sensor may be provided in a position corresponding to the user's sole. Therefore, the lengths of the user's legs may be precisely measured according to the change in lengths of the leg massage units 2000a and 2000b.

Below, the method of operating the above-described massage device 1000 will be described in detail.

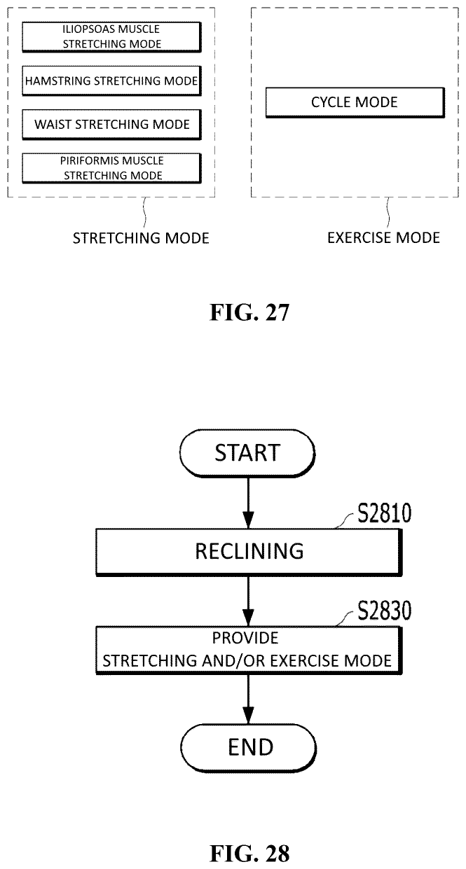

The method of operating the massage device 1000 according to the present invention includes receiving a user input, recognizing body information about at least a part of the user's body, adjusting the massage device based on the body information, and providing a massage to a user using the adjusted massage device. FIG. 27 is a diagram for describing a stretching mode and an exercise mode provided by the massage device according to an embodiment of the present disclosure.

The massage device 1000 may provide a stretching mode for stretching the user's body and a leg rehabilitation mode (or cycle mode) for exercising the user's legs. In this case, the stretching mode may be a mode for stretching at least one of the user's legs, a waist, and buttocks (e.g., hip area). The cycle mode may be a mode used by users with normal legs to exercise their legs, or a mode used by users with abnormal legs due to various injuries such as knee injuries to rehabilitate their legs.

For example, the stretching mode may include at least one of a iliopsoas muscle stretching mode (or iliopsoas muscle relaxation mode) that stretches iliopsoas muscles of legs, a hamstring stretching mode (or hamstring relaxation mode) that stretches hamstrings of legs, a waist stretching mode (or rolling relaxation mode) that stretches a waist, and a piriformis muscle stretching mode (piriformis muscle relaxation mode) that stretches piriformis muscles of buttocks.

Specifically, the controller 1400 may execute each stretching mode and/or cycle mode based on the user's response, or may automatically execute each stretching mode and/or cycle mode. In this case, the user's response may be a response (or signal) for executing the stretching mode and/or the cycle mode.

The controller 1400 may provide each stretching mode and/or cycle mode to the user by controlling the operation of the massage device 1000 according to massage maps for each stretching mode and/or cycle mode. In this case, the massage map may be preset from various objects and stored in the memory 1430. Objects may be various, such as users and administrators who use the massage devices, and/or workers and doctors from various companies such as manufacturers, sellers, distributors, and developers of the massage device.

As an example, the controller 1400 may provide the iliopsoas muscle stretching mode to the user by controlling the operation of the massage device 1000 based on the massage map for the iliopsoas muscle stretching mode. In this way, the controller 1400 may relax the iliopsoas muscle by stretching the user's iliopsoas muscle and thus, straighten the bent back and reduce excessive stress on the lumbar spine, thereby alleviating back pain.

As another example, the controller 1400 may provide the hamstring stretching mode to the user by controlling the operation of the massage device 1000 based on the massage map for the hamstring stretching mode. In this way, the controller 1400 may prevent shortening of muscles and injuries by stretching the user's hamstrings and relaxing the hamstring muscles.

As still another example, the controller 1400 may provide the waist stretching mode to the user by controlling the operation of the massage device 1000 based on the massage map for the waist stretching mode. In this way, the controller 1400 may relax the muscles around the lumbar spine by stretching the user's waist, thereby improving the mobility of the muscles around the lumbar spine and reducing the back pain. As still another example, the controller 1400 may provide the piriformis muscle stretching mode to the user by controlling the operation of the massage device 1000 based on the massage map for the piriformis muscle stretching mode. In this way, the controller 1400 stretches the user's buttocks (or piriformis muscle) to relax the muscles (or piriformis muscle) around the buttocks, thereby reducing the waist and buttock pain, leg numbness, sciatica, etc., and improving hip joint mobility in the lower body.

The controller 1400 may provide the cycle mode to the user by controlling the operation of the massage device 1000 based on the massage map for the cycle mode. In this way, the massage device 1000 may exercise the normal leg to keep the normal leg in a healthy state, and perform rehabilitation exercises for the abnormal leg to restore the abnormal leg to a healthy state. The massage device 1000 may prevent joint contractures by maintaining and improving the flexibility and movement of joints (pelvis, knees, ankles) by exercising the legs (or rehabilitating the legs) through repetitive joint movements. In other words, the cycle mode may be both a leg exercise mode and a leg rehabilitation mode (or knee rehabilitation mode).

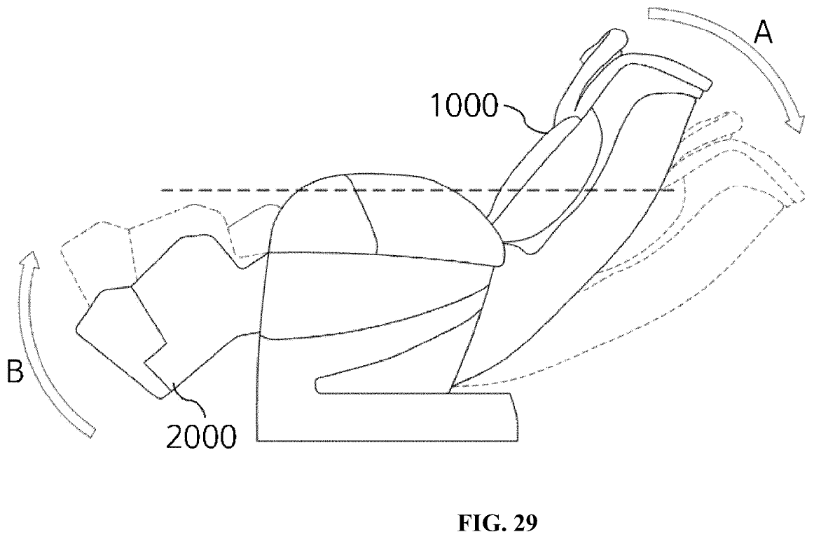

FIG. 28 illustrates a diagram for describing an example of the massage mode illustrated in FIG. 27, and FIG. 29 illustrates an example for describing a reclining operation of a controller according to an embodiment of the present disclosure.

Referring to FIGS. 28 and 29, the controller 1400 may recline the massage device 1000 (S2810) and provide the stretching mode and/or exercise mode to the user while the massage device 1000 is reclining (S2830).

First, the controller 1400 may tilt the body massage unit 1100 toward the rear of the massage device 1000 and tilt (or rotate) each leg massage unit in the up direction of the massage device 1000 to recline the massage device 1000 (S2810). In the reclined massage device 1000, the body massage unit 1100 may be tilted in the rear direction of the massage device 1000, and the leg massage unit 2000 may rotate in the up direction of the massage device 1000.

The controller 1400 may adjust the length of each leg massage unit based on the lengths of each leg while tilting each leg massage unit. Since the length adjustment operation of each leg massage unit is the same as described above, a detailed description thereof will be omitted.

Thereafter, the controller 1400 may provide the stretching mode and/or exercise mode to the user by controlling the operation of the massage device 1000 according to the stretching mode and/or the exercise mode while the massage device 1000 is reclining (S2830). For example, the controller 1400 may perform the operation corresponding to the stretching mode and/or an exercise mode while the massage device 1000 is relining.

In this way, the controller 1400 may secure a space for stretching the user's legs and a space for exercising the user's legs by reclining the massage device 1000 to eliminate space limitations for stretching the user's legs and space limitations for exercising the legs, thereby increasing the leg stretching effect and leg exercise effect.

In addition, the controller 1400 may provide a relaxation massage that relaxes the user's body through the massage unit (e.g., at least one of the body massage unit 1100, the arm massage unit 1200, and the leg massage unit 2000) while the massage device 1000 is reclining, and then provide the stretching mode and/or exercise mode. In this case, the relaxation massage may be a massage according to a preset massage intensity and speed. For example, the relaxation massage may be provided at the average massage intensity and average massage speed among the massage intensities and speeds that the massage device 1000 can provide, or may be provided at the lowest massage intensity and lowest massage speed. In this case, the controller 1400 may relax the user's body to prevent injuries due to the stretching mode and/or exercise mode, and increase the leg stretching effect and leg exercise effect.

As described above, the controller 1400 reclines the massage device 1000, provides the relaxation massage, and then provides the stretching mode and/or cycle mode to the user, but is not limited thereto. For example, the controller 1400 may provide the stretching mode and/or exercise mode while performing at least one of an operation of reclining the massage device 1000 and an operation of providing the relaxation massage.

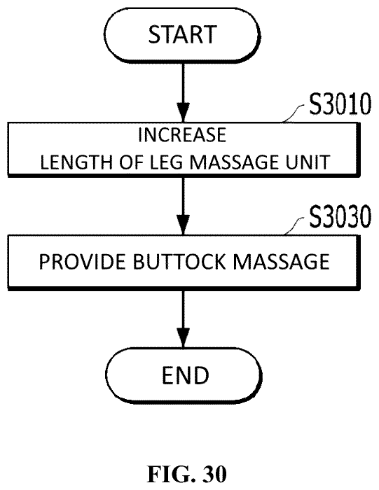

FIG. 30 is a flowchart for describing a piriformis muscle stretching mode illustrated in FIG. 27.

The controller 1400 may provide the piriformis muscle stretching mode to the user by controlling the operation of the massage device 1000 with the operation corresponding to the piriformis muscle stretching mode.

First, the controller 1400 may stretch the user's legs by increasing the length of any one of the first leg massage unit 2000a and the second leg massage unit 2000b through the leg length adjustment module 2500 (S3010).

The controller 1400 may fix the user's legs by expanding an air cell of any one leg massage unit while increasing the length of one leg massage unit. For example, the controller 1400 may expand a leg air cell included in at least one of the upper leg massage unit and the lower leg massage unit of any one leg massage unit. Specifically, the controller 1400 may pre-expand a foot air cell (e.g., at least one of the first and second sub-foot air cells, etc.) included in the lower leg massage unit of any one leg massage unit, and then post-expand a calf air cell (e.g., at least one of the first and second sub-calf air cells, etc.) included in the upper leg massage unit of any one leg massage unit.

As described above, the controller 1400 expands the air cell of any one leg massage unit while increasing the length of one leg massage unit, but is not limited thereto. For example, the controller 1400 may expand the air cell of any one leg massage unit and then increase the length of any one leg massage unit.

In this way, the controller 1400 may sufficiently extend the piriformis muscle of the leg by pulling (tracting) the leg to be stretched, which is the leg corresponding to any one leg massage unit of either the one leg or the other leg of the user. In this case, the controller 1400 may accurately extend the leg to be stretched by accurately holding the lower portion of the leg to be stretched and pulling the leg to be stretched, compared to the case where the lower portion of the leg to be stretched is not fixed. In addition, the controller 1400 may additionally hold the upper portion of the leg to be stretched and continuously pull the leg to be stretched, thereby improving the stretching effect on the leg to be stretched.

The controller 1400 may provide the massage to the user's buttocks by moving the massage module 1110 to the user's buttocks (S3030). For example, the controller 1400 may move the massage module 1110 to an area corresponding to the user's buttocks among the entire area of the main frame 1130. The controller 1400 may massage the user's buttocks by protruding the massage module 1110, which has moved to the user's buttocks, in the front direction of the massage module 1110. In this case, the front direction of the massage module 1110 may be the direction in which the massage module 1110 approaches the user's body. In other words, the controller 1400 may provide an acupressure massage to the user's buttocks through the massage module 1110.

In this way, the controller 1400 may sufficiently relax the piriformis muscle by providing the massage to the extended piriformis muscle through the massage module 1110.

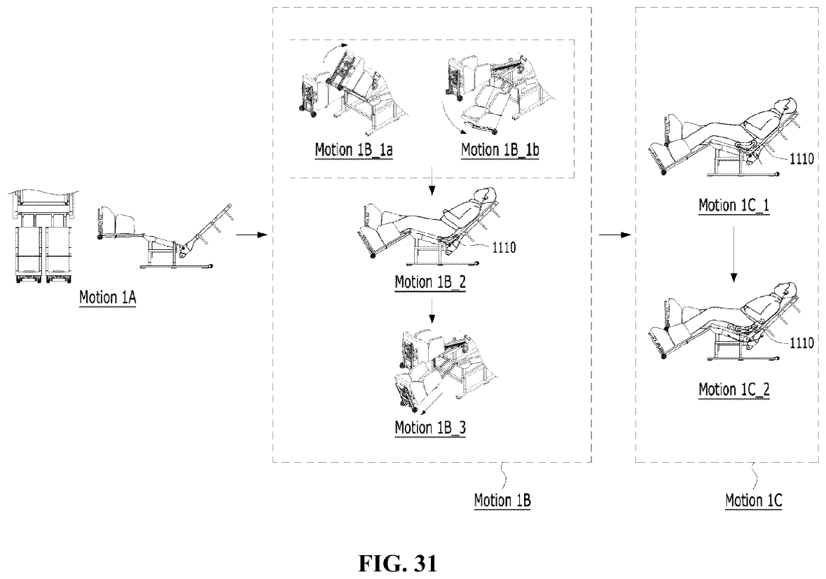

FIG. 31 is a diagram for describing each operation of the piriformis muscle stretching mode illustrated in FIG. 30.

The controller 1400 may acquire the user's shoulder position through the massage module 1110 and then recline the massage device 1000 (or after reclining the massage device 1000, the user's shoulder position may be acquired through the massage module 1110 (Motion 1A). In this case, as described above, the controller 1400 may adjust the lengths of each leg massage unit while the massage device 1000 is reclining (or after the reclining operation is performed) based on the length of each leg.

After performing Motion 1A, the controller 1400 may increase the length of one of the first leg massage unit 2000a and the second leg massage unit 2000b through the leg length adjustment module 2500 while the massage device 1000 is reclining (Motion 1B).

First, before increasing the length of any one leg massage unit, the controller 1400 may adjust (or change) the angle of any one leg massage unit through the leg angle adjustment module 1350 while the massage device 1000 is reclining (Motion 1B_1).

For example, the controller 1400 may operate (or drive) the leg angle adjustment module corresponding to one of the first and second leg angle adjustment modules 1350a and 1350b to adjust the angle of any one leg massage unit.

Specifically, the controller 1400 tilts one leg massage unit in at least one of clockwise and counterclockwise directions through the leg angle adjustment module corresponding to one leg massage unit to adjust the angle of any one leg massage unit. In this case, the controller 1400 may disable the remaining leg angle adjustment modules that are different from the leg angle adjustment module corresponding to the leg massage unit of one of the first and second leg angle adjustment modules 1350a and 1350b. In this way, the controller 1400 may fix the location of the remaining leg massage unit that is different from the leg massage unit of one of the first and second leg massage units 2000a and 2000b.

As an example, the controller 1400 maximally extends the length of the leg angle adjustment module corresponding to any one leg massage unit to raise any one leg massage unit to the maximum, thereby maximally increasing the angle of any one leg massage unit (Motion 1B_1a).

As another example, the controller 1400 maximally shortens the length of the leg angle adjustment module corresponding to any one leg massage unit to lower any one leg massage unit to the maximum, thereby maximally lowering the angle of any one leg massage unit (Motion 1B_1b).

In this way, the controller 1400 may flex a user's hip joint before extending the user's legs and prevent injuries to the body from occurring by extending the user's legs.

Thereafter, before increasing the length of any one leg massage unit, the controller 1400 may move the massage module 1110 to the positions corresponding to the user's buttocks while the angle of any one leg massage unit is adjusted, and massage the user's buttocks through the massage module 1110 (Motion 1B_2).

For example, the controller 1400 may predict the position of the user's buttocks based on the user's shoulder position. The controller 1400 moves the massage module 1110 to the user's buttocks based on the predicted positions of the user's buttocks, thereby disposing the massage module 1110 on the user's buttocks. As described above, the controller 1400 moves the massage module 1110 based on the predicted positions of the user's buttocks, but is not limited thereto. For example, the controller 1400 may move the massage module 1110 based on the preset positions of the user's buttocks.

The controller 1400 may provide the massage to the user's buttocks through the massage module 1110 disposed on the user's buttocks. In this case, the massage may be a variety of massages (e.g., acupressure massage, kneading massage, complex massage, etc.) to reduce tension in the user's buttocks, but hereinafter, for convenience of description, it will be assumed to be the tapping massage. The tapping massage may be preset to various intensities and speeds to provide a gentle massage such as providing at the lowest intensity and lowest speed among the massage intensities and speeds that the massage module 1110 can provide, or providing at the average intensity and average speed.

In this way, the controller 1400 may provide the massage that reduces tension in the muscles of the buttocks before extending and relaxing the muscles of the user's buttocks (e.g., the piriformis muscle), thereby preventing the user's injury and side effects from stretching the piriformis muscle.

As described above, the controller 1400 moves the massage module 1110 while the angle of any one leg massage unit is adjusted, but is not limited thereto. For example, the controller 1400 may move the massage module 1110 while the angle of any one leg massage unit is adjusted, or move the massage module 1110 (or while the massage module 1110 moves), and then adjust the angle of any one leg massage unit.

Finally, after ending the Motion 1B_2 operation, the controller 1400 may increase the length of any one leg massage unit through the leg length adjustment module 2500 (Motion 1B_3). In this case, the controller 1400 may stop the operation of the massage module 1110 and end the Motion 1B_2 operation. Before stopping the operation of the massage module 1110, the controller 1400 may move the massage module 1110 toward the rear of the massage module 1110 as far as possible and then stop the massage module 1110.

For example, the controller 1400 operates (or drives) the leg length adjustment module corresponding to the leg massage unit of any one of the first and second leg length adjustment modules 2500a and 2500b to increase the length of any one leg massage unit. In this case, as described above, the controller 1400 may expand the air cell of any one leg massage unit.

Specifically, the controller 1400 may increase the length of any one leg massage unit by moving any one leg massage unit toward the down direction of any one leg massage unit through the leg length adjustment module corresponding to any one leg massage unit. In this case, the controller 1400 may disable the remaining leg length adjustment module that is different from the leg length adjustment module corresponding to the leg massage unit of any one of the first and second leg length adjustment modules 2500a and 2500b. In this way, the controller 1400 may fix the length of the remaining leg massage unit that is different from the leg massage unit of any one of the first and second leg massage units 2000a and 2000b.

Hereinafter, the leg length adjustment operation will be described in detail.

The controller 1400 may increase the length of any one leg massage unit by moving any one leg massage unit in the down direction of any one leg massage unit based on the reference length for increasing the length of any one leg massage unit.

For example, the reference length is a reference indicating the degree of pulling the leg to be stretched and may mean a movement length (or movement distance) to move any one leg massage unit in the down direction of any one leg massage unit. The reference length is a length at which the leg to be stretched may be sufficiently extended and may be preset by various objects and stored in the memory 1430. For example, the reference length may be set variously, such as about 5 cm to 10 cm.

As described above, the reference length may be preset, but is not limited thereto. For example, the controller 1400 may determine a customized reference length customized to the user as the reference length based on the length of the leg corresponding to the leg massage unit of any one of the one leg and the other leg of the user. Specifically, the memory 1430 may store a matching table in which each of the arbitrary leg lengths and each of the arbitrary reference lengths are matched to each other. The controller 1400 may compare the lengths of the user's legs with the matching table and determine arbitrary reference length corresponding to the length of the leg among the arbitrary reference lengths included in the matching table as the customized reference length. The controller 1400 may determine the customized reference length as the reference length to adjust (or increase) the length of any one leg massage unit. The controller 1400 may increase the length of any one leg massage unit based on the reference length customized to the user.

As described above, the controller 1400 increases the length of any one leg massage unit after ending the Motion 1B_2 operation which is the massage operation for massaging the user's buttocks, but is not limited thereto. For example, after ending the Motion 1B_2 operation, the controller 1400 may increase the length of any one leg massage unit while massaging at least a part of the user's body.

Specifically, the controller 1400 may end the Motion 1B_2 operation, change the location of the massage module 1110, and provide the massage to at least a part of the user's body. In this case, the controller 1400 may provide the massage through the massage module 1110 to body parts of the user's body excluding the user's buttocks. The controller 1400 may increase the length of any one leg massage unit after providing the massage through the massage module 1110 or while providing the massage.

In addition, the controller 1400 may fix the user's pelvis by expending the pelvic air cells of the body massage unit 1100 to a preset expansion level while increasing the length of any one leg massage unit. In this case, the pelvic air cell may be disposed in the area corresponding to the user's pelvis, which is both side portions of the area where the user sits, in the entire area of the massage device 1000.

As described above, the controller 1400 expands the pelvic air cell while increasing the length of any one leg massage unit, but is not limited thereto. For example, controller 1400 may expand the pelvic air cell before increasing the length of any one leg massage unit.

In this way, the controller 1400 may fix the user's legs and pelvis and extend the leg to be stretched longer to extend the leg to be stretched more accurately than when the user's pelvis is not fixed, thereby improving the stretching effect on the leg to be stretched.

As described above, the controller 1400 sequentially performs the Motion 1B_1, 1B_2, and 1B_3, but is not limited thereto. For example, the controller 1400 may freely perform Motion 1B_1, 1B_2, and 1B_3 by controlling (or randomly) the operation order of the Motion 1B_1, 1B_2, and 1B_3, or perform Motion 1B_1, 1B_2, and 1B_3 simultaneously.

When the Motion 1B is performed, the controller 1400 may provide the massage to the user's buttocks through the massage module 1110 (Motion 1C). In this case, the controller 1400 may drive the massage module 1110 when the operation of the massage module 1110 is stopped, and may move the massage module 1110 to the user's buttocks when the position of the massage module 1110 does not correspond to the user's buttocks to provide the massage to the user's buttocks.

First, the controller 1400 may massage the user's buttocks by protruding the massage module 1110 in the front direction of the massage module 1110 based on the reference intensity for massaging the user's buttocks (Motion 1C_1). In this case, the reference intensity may refer to the degree of protrusion of the massage module 1110 as the reference representing the massage intensity of acupressuring the user's buttocks. The reference intensity is the massage intensity that may sufficiently relax the user's buttocks and may be preset by various objects and stored in the memory 1430. For example, the reference intensity may be set variously, such as the intensity that causes the massage module 1110 to protrude about 4 cm to 8 cm.

As described above, the reference intensity may be preset, but is not limited thereto. For example, the controller 1400 may determine the customized reference intensity customized to the user as the reference intensity based on various information such as at least one of the user's biometric information, the massage device use history, and user preference information.

Thereafter, the controller 1400 may additionally massage the user's buttocks by reciprocating the massage module 1110 while the massage module 1110 protrudes (Motion 1C_2). In this case, the controller 1400 may repeatedly provide the additional massage that additionally massages the user's buttocks by repeating the reciprocating motion. The reciprocating motion may mean an operation of rubbing and massaging the user's buttocks through the massage module 1110.

For example, the controller 1400 may move the massage module 1110 in the up direction of the massage module 1110 by a preset first distance and then move the massage module 1110 in the down direction of the massage module 1110 by a preset second distance to reciprocate the massage module 1110. The first and second distances may be preset by various objects and stored in the memory 1430. The first and second distances may be set to be different from or equal to each other. The up direction of the massage module 1110 may be a direction from the lower side of the massage module 1110 toward the upper side of the massage module 1110, and the down direction of the massage module 1110 may be a direction from the upper side of the massage module 1110 toward the lower side of the massage module 1110.

As described above, the controller 1400 reciprocates the massage module 1110 in the up and down directions of the massage module 1110, but is not limited thereto. For example, the controller 1400 may reciprocate the massage module 1110 in the front and rear directions of the massage module 1110. In this case, the front direction of the massage module 1110 may be a direction from the rear of the massage module 1110 toward the front of the massage module 1110, and the rear direction of the massage module 1110 may be a direction from the front of the massage module 1110 toward the rear of the massage module 1110. The front of the massage module 1110 may be a portion disposed adjacent to the user and in contact with the user's body.

Specifically, the controller 1400 may additionally protrude the massage module 1110 in the front direction of the massage module 1110 by a first distance while the massage module 1110 protrudes in front of the massage module 1110, and then retract in the rear direction of the massage module 1110 by a second distance to reciprocate the massage module 1110.

When additionally massaging the user's buttocks through the reciprocating motion of the massage module 1110 described above, the controller 1400 may reduce the length of any one leg massage unit by a preset length and expand the pelvic air cells of the body massage unit 1100 to the maximum to strongly fix the user's pelvis. For example, the controller 1400 may expand the pelvic air cell to the maximum while reciprocating the massage module 1110 or before reciprocating the massage module 1110.

In this way, the controller 1400 may cause the additional massage additionally massaging the user's buttocks to be precisely provided to the user's buttocks by strongly fixing the user's pelvis even while reciprocating the massage module 1110.

As described above, the controller 1400 performs the piriformis muscle stretching motions (Motions 1A, 1B, and 1C) through any one leg massage unit, but is not limited thereto. For example, the controller 1400 may perform the piriformis muscle stretching motions (Motions 1A, 1B, and 1C) through any one leg massage unit, and then additionally perform the piriformis muscle stretching motions for the remaining leg massage units. In this case, the piriformis muscle stretching motion for the remaining leg massage unit may be the same as the Motions 1A, 1B, and 1C.

In addition, the controller 1400 may sequentially and repeatedly perform the piriformis muscle stretching motion for each leg massage unit described above.

In this way, the controller 1400 may sufficiently stretch and relax the piriformis muscle of the user by stretching both legs and both buttocks of the user in a balanced manner and overall relaxing the user's legs and buttocks by stretching repeatedly.

In addition, the controller 1400 may provide a thermal massage by increasing the temperature of the massage module 1110 while providing the massage to the user's buttocks through the massage module 1110 while performing the piriformis muscle stretching motion.

In this way, the controller 1400 may provide a thermal sensation to the user's buttocks and extend and relax the user's buttocks, thereby extending and relaxing the user's buttocks more than when it does not provide the thermal sensation, and preventing injuries due to the extension and relaxation.

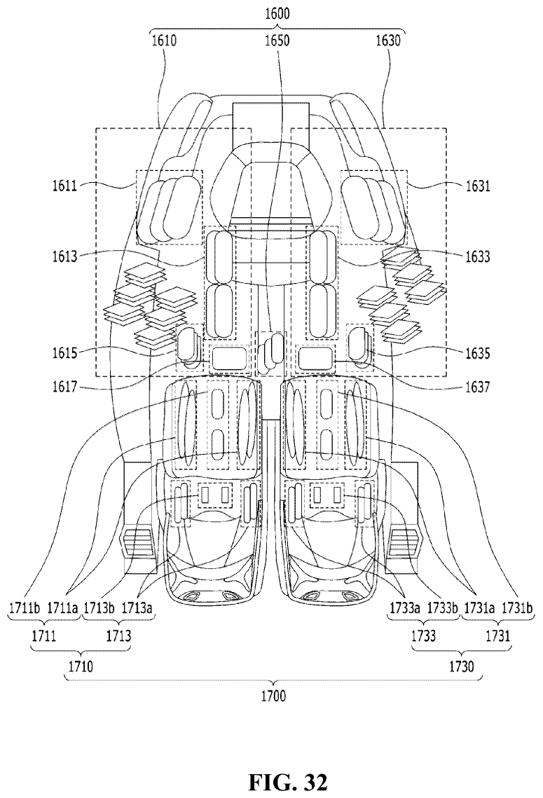

FIG. 32 is a diagram for describing a body air cell and a leg air cell included in a body massage unit according to an embodiment of the present disclosure.

The body massage unit 1100 may include a body air cell 1600 that massages (or presses) the user's body.

For example, the body air cell 1600 may include at least one of a shoulder air cell corresponding to the user's shoulder, a back air cell corresponding to the user's back, a pelvic air cell corresponding to the user's pelvis, a thigh air cell corresponding to the user's thigh, and a perineum air cell corresponding to the user's perineum.

Specifically, the body air cell 1600 may include a first body air cell 1610 that is disposed on one side of the body massage unit 1100 to massage one side of the user's body, a second body air cell 1630 that is disposed on the other side of the body massage unit 1100 to massage the other side of the user's body, and a third body air cell 1650 that is disposed between one side and the other side of the body massage unit 1100 to massage one side and the other side of the user's body. The first body air cell 1610 may be an air cell corresponding to the first leg massage unit 2000a, and the second body air cell 1610 may be an air cell corresponding to the second leg massage unit 2000b.

The first body air cell 1610 may include at least one of a first shoulder air cell 1611, a first back air cell 1613, a first pelvic air cell 1615, and a first thigh air cell 1617, and the second body air cell 1630 may include at least one of a second shoulder air cell 1631, a second back air cell 1633, a second pelvic air cell 1635, and a second thigh air cell 1637.

The first shoulder air cell 1611 may be disposed in an area (e.g., an upper area of one side of the body massage unit 1100) corresponding to one side of the user's shoulder in the entire area of one side of the body massage unit 1100 to massage one side of the user's shoulder. The second shoulder air cell 1631 may be disposed in an area (e.g., an upper area of the other side of the body massage unit 1100) corresponding to the other side of the user's shoulder in the entire area of the other side of the body massage unit 1100 to massage the other side of the user's shoulder. In this case, the first and second shoulder air cells 1611 and 1631 may massage (or fix) the user's shoulders by pressing both side surfaces (or upper surfaces of both sides of the shoulders) of the user's shoulders.

The first back air cell 1613 may be disposed in an area (e.g., an area between an upper end of one side and a lower end of one side of the body massage unit 1100) corresponding to one side of the user's back in the entire area of one side of the body massage unit 1100 to massage one side of the user's back. The second back air cell 1633 may be disposed in an area (e.g., an area between an upper end of the other side and a lower end of the other side of the body massage unit 1100) corresponding to the other side of the user's back in the entire area of the other side of the body massage unit 1100 to massage the other side of the user's back. In this case, the first and second back air cells 1613 and 1633 may press the rear surface of the user's back to massage the user's back, thereby pushing (or moving) the user's back toward the front of the body massage unit 1100.

The first pelvic air cell 1615 may be disposed in an area (e.g., a lower area of one side of the body massage unit 1100) corresponding to one pelvis of the user in the entire area of one side of the body massage unit 1100 to massage one pelvis of the user. The second pelvic air cell 1635 may be disposed in an area (e.g., a lower area of the other side of the body massage unit 1100) corresponding to the other pelvis of the user in the entire area of the other side of the body massage unit 1100 to massage the other pelvis of the user. In this case, the first and second pelvic cells 1615 and 1635 may massage (or fix) the user's pelvis by pressing both side surfaces of the user's pelvis.

The first thigh air cell 1617 may be disposed in an area (or one central area in the lower central area of the body massage unit 1100) corresponding to the thigh of one leg of the user in the entire area of one side of the body massage unit 1100 to massage the thigh of one leg of the user. The second thigh air cell 1637 may be disposed in the area (or the other central area in the lower central area of the body massage unit 1100) corresponding to the thigh of the other leg of the user in the entire area of the other side of the body massage unit 1100 to massage the thigh of the other leg of the user. In this case, the first and second thigh air cells 1617 and 1637 may press the rear surface of the user's thigh to massage one thigh and the other thigh of the user, thereby pushing the user's thigh in the up direction of the body massage unit 1100.

The third body air cell 1650 may be disposed in an area (e.g., the front center area of the body massage unit 1100) corresponding to the user's perineum in the entire area between one side and the other side of the body massage unit 1100 to massage the user's perineum. In this case, the perineum air cell, which is the third body air cell 1650, may press the user's perineum and push the user's body in the up direction of the body massage unit 1100.

In addition, the leg massage unit 2000 may include a leg air cell 1700 that massages the user's legs.

For example, the leg air cell 1700 may include a first leg air cell 1710 that corresponds to the first leg massage unit 2000a (or one leg of the user), and a second leg air cell 1730 that corresponds to the second leg massage unit 2000b (or the other leg of the user).

Specifically, the first leg massage unit 2000a may include a first leg air cell 1710 that is disposed in at least a partial area of the first leg massage unit 2000a, and the second leg massage unit 2000b may include a second leg air cell 1730 that is disposed in at least a partial area of the second leg massage unit 2000b.

Each leg air cell 1710 and 1730 may include at least one of the calf air cell corresponding to the user's calf and the foot air cell corresponding to the user's foot.

For example, the first leg air cell 1710 may include at least one of a first calf air cell 1711 and a second foot air cell 1713, and the second leg air cell 1730 may include at least one of a second calf air cell 1731 and a second foot air cell 1733.

The first calf air cell 1711 may be disposed in an area (e.g., the upper area of the first leg massage unit 2000a) corresponding to the calf of one leg of the user in the entire area of the first leg massage unit 2000a to massage the user's calf.

For example, the first calf air cell 1711 may include at least one of a first sub-calf air cell 1711a that is disposed in an area corresponding to the calf side portion of one leg to massage (or press) the calf side portion, and a second sub-calf air cell 1711b that is disposed in an area corresponding to a rear of the calf of one leg to massage the rear of the calf. In this case, the first sub-calf air cell 1711a may massage (or fix) the user's calf by pressing both side portions of the calf of one leg.

Since the second calf air cell 1731 is the same as the above-described first calf air cell 1711 applied to the second leg massage unit 2000b, a detailed description will be omitted.

The first foot air cell 1713 may be disposed in an area (e.g., the lower area of the first leg massage unit 2000a) corresponding to the foot of one leg of the user in the entire area of the second leg massage unit 2000a to massage the user's feet.

For example, the first foot air cell 1713 may include at least one of a first sub-foot air cell 1713a that is disposed in an area corresponding to a foot side portion (or instep) of one leg to massage (or press) the foot side portion (or instep) and a second sub-foot air cell 1713b that is disposed in an area corresponding to a rear (or heel, Achilles tendon) of the foot of one leg to massage (or press) the rear (or heel, Achilles tendon) of the foot. In this case, the first sub-foot air cell 1713a may massage (or fix) the user's foot by pressing both side portions of the foot of one leg.

Since the second foot air cell 1733 is the same as the above-described first foot air cell 1713 applied to the second leg massage unit 2000b, a detailed description will be omitted.

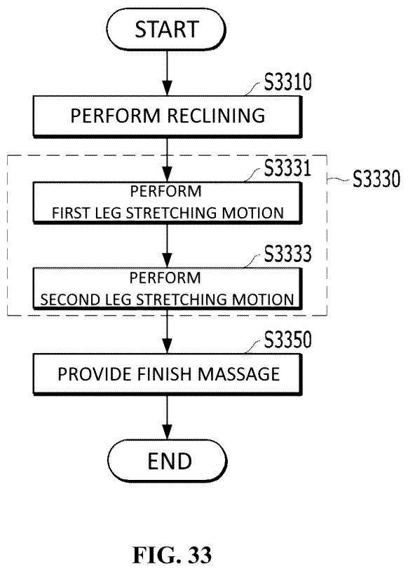

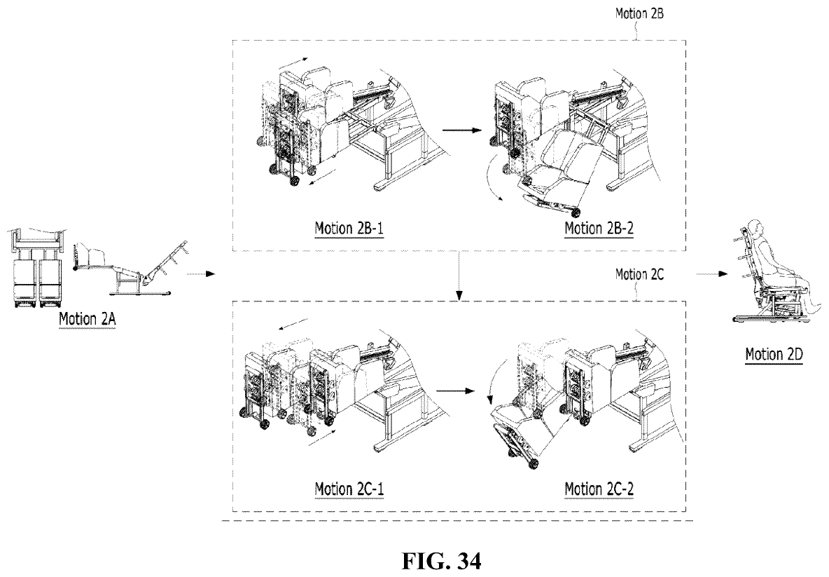

FIG. 33 illustrates a flowchart for describing the iliopsoas muscle stretching mode illustrated in FIG. 27, and FIG. 34 illustrates a diagram for describing each operation illustrated in FIG. 33.

The controller 1400 may provide the user with the iliopsoas muscle stretching mode that stretches the iliopsoas muscles of the user's legs by controlling the operation of the massage device 1000 with the operation corresponding to the iliopsoas muscle stretching mode. In this case, the iliopsoas muscle stretching mode may include the leg stretching motion that stretches the user's legs (or the iliopsoas muscle of the leg).

For example, the controller 1400 may recline the massage device 1000 like Motion 2A before performing the leg stretching motion (S3310).

Specifically, the controller 1400 may tilt (or while tilting) the body massage unit 1100 toward the rear of the body massage unit 1100 based on the preset body angle, and then adjust the angle of the leg massage unit 2000 by tilting the leg massage unit 2000 in the up direction of the massage device 1000 based on the preset leg angle.

The body angle and the leg angle may be preset from various objects and stored in the memory 1430. The body angle is any one of a minimum body angle or a maximum body angle between the ground and the body massage unit 1100, and may be greater than the minimum body angle and smaller than the maximum body angle. The minimum body angle may be the maximally tilted angle of the body massage unit 1100 in the front direction of the body massage unit 1100. The minimum body angle may be the maximally tilted angle of the body massage unit 1100 in the rear direction of the body massage unit 1100. The leg angle may be any one of the minimum leg angle and the maximum leg angle between the body massage unit 1100 and each leg massage unit, and may be an angle greater than the minimum leg angle and smaller than the maximum leg angle. The minimum leg angle may be the maximally tilted angle of each leg massage unit in the down direction of the massage device 1000. The maximum leg angle may be the maximally tilted angle of each leg massage unit in the up direction of the massage device 1000.

The controller 1400 may perform a preliminary operation including at least one of the operation of providing the relaxation massage and the operation of measuring the leg length while the massage device 1000 is reclining.

For example, the controller 1400 may provide (or while providing) the relaxation massage to relax the user's body and measure the lengths of the user's legs.

Since the motion of providing the relaxation massage and the motion of measuring the leg length are the same as described above, detailed descriptions will be omitted. However, the controller 1400 can move the massage module 1110 to the positions corresponding to the user's buttocks while providing the relaxation massage. Since the contents described in the Motion 1B_2 can be applied to the massage module movement operation, a detailed description will be omitted.

The controller 1400 may perform the leg stretching motion while the massage device 1000 is reclining through the leg length adjustment module 2500 and the leg angle adjustment module 1350 (S3330).

The leg stretching motion may include the first leg stretching motion and/or the second leg stretching motion. The first leg stretching motion may stretch one leg of the user, thereby stretching the iliopsoas muscle of one leg. The second leg stretching motion may stretch the other leg of the user, thereby stretching the iliopsoas muscle of the other leg.

For example, the controller 1400 may sequentially and repeatedly perform the first and second leg stretching motions based on a preset number of times of repetitions. In this case, the number of times of repetitions may be preset from various objects and stored in the memory 1430. Hereinafter, for convenience of description, it will be assumed that the first and second leg stretching motions are not repeatedly performed.

First, the controller 1400 may perform the first leg stretching motion like Motion 2B (S3331).

For example, the controller 1400 may increase the length of the first leg massage unit 2000a while adjusting or maintaining the length of the second leg massage unit 2000b, such as Motion 2B-1. In this case, the length of the first leg massage unit 2000a may mean the length from one end to the other end of the first leg massage unit 2000a. The length of the second leg massage unit 2000b may mean the length from one end to the other end of the second leg massage unit 2000b.

Specifically, the controller 1400 may determine the adjustment length to adjust the length of the first leg massage unit 2000a based on the length of one leg of the user. The controller 1400 may adjust the length of the first leg massage unit 2000a based on the adjustment length. The length adjustment operation of the first leg massage unit 2000a will be described in detail below.

Hereinafter, for convenience of description, it will be assumed that the length of the first leg massage unit 2000a is adjusted while the length of the second leg massage unit 2000b is adjusted during the first leg stretching motion.

The controller 1400 may adjust the angle of the first leg massage unit 2000a while maintaining the angle of the second leg massage unit 2000b, like Motion 2B-2. In this case, the angle of the first leg massage unit 2000a may mean the angle between the first leg massage unit 2000a and the body massage unit 1100 (or the seat part of the body massage unit). The angle of the second leg massage unit 2000b may mean the angle between the second leg massage unit 2000b and the body massage unit 1100.

For example, the controller 1400 may determine the adjustment angle to adjust the angle of the first leg massage unit 2000a based on the length of one leg of the user. The controller 1400 may adjust the angle of the first leg massage unit 2000a based on the adjustment angle. The angle adjustment operation of the first leg massage unit 2000a will be described in detail below.

The controller 1400 may restore the position of the leg massage unit 2000 after performing the first leg stretching motion. The restored angle and length of the leg massage unit 2000 may be the angle and length of the leg massage unit 2000 before the first leg stretching motion is performed.

Thereafter, the controller 1400 may perform a second leg stretching motion such as Motion 2C (S3333).

For example, the controller 1400 may increase the length of the second leg massage unit 2000b while adjusting or maintaining the length of the first leg massage unit 2000a, such as Motion 2C-1.

Specifically, the controller 1400 may determine the adjustment length to adjust the length of the second leg massage unit 2000b based on the length of the other leg of the user. The controller 1400 may adjust the length of the second leg massage unit 2000b based on the adjustment length. As the length adjustment operation of the second leg massage unit 2000b, the length adjustment operation of the first leg massage unit 2000a included in the first leg stretching motion, which will be described later, may be applied.

Hereinafter, for convenience of description, it will be assumed that the length of the second leg massage unit 2000b is adjusted while the length of the first leg massage unit 2000a is adjusted during the second leg stretching motion.

The controller 1400 may adjust the angle of the second leg massage unit 2000b while maintaining the angle of the first leg massage unit 2000a, like Motion 2C-2.

For example, the controller 1400 may determine the adjustment length to adjust the angle of the second leg massage unit 2000b based on the length of the other leg of the user. The controller 1400 may adjust the angle of the second leg massage unit 2000b based on the adjustment angle. As the angle adjustment operation of the second leg massage unit 2000b, the angle adjustment operation of the first leg massage unit 2000a included in the first leg stretching motion, which will be described later, may be applied.

The controller 1400 may restore the position of the leg massage unit 2000 after performing the second leg stretching motion.

After performing the leg stretching motion, the controller 1400 may perform a finish massage providing operation to stretch the user's legs and buttocks through the leg length adjustment module 2500, the leg angle adjustment module 1350, and the massage module 1110 like Motion 2D (S3350).

For example, the controller 1400 may reduce the length of each leg massage unit while tilting each leg massage unit in the up direction of the massage device 1000 while the leg massage unit 2000 is restored to its original position. For example, the controller 1400 may adjust the angles and lengths of each leg massage unit based on the adjustment angle and the adjustment angle according to the lengths of each leg. As another example, the controller 1400 may adjust the angles of each leg massage unit to the maximum leg angle and reduce the lengths of each leg massage unit to the minimum length.

The controller 1400 may provide the massage to the user's buttocks through the massage module 1110 while the angles and lengths of each leg massage unit are adjusted. In this case, the massage provided to the buttocks may be the buttock massage in the piriformis muscle stretching mode, such as at least one of acupressure massage, kneading massage, tapping massage, and combined massage.

The controller 1400 may end the iliopsoas muscle stretching mode after providing the massage.

The length adjustment operations of each leg massage unit included in the above-described first and second leg stretching motions may be performed simultaneously, at a time interval, or sequentially. Hereinafter, for convenience of description, it is assumed that the length adjustment operation of each leg massage unit is performed simultaneously.

In the following, since the first leg stretching motion can be applied to the second leg stretching motion, for convenience of description, only the first leg stretching motion will be described. For example, the operation of the first leg massage unit 2000a included in the first leg stretching motion may be applied to the operation of the second leg massage unit 2000b included in the second leg stretching motion. The operation of the second leg massage unit 2000b included in the first leg stretching motion may be applied to the operation of the first leg massage unit 2000a included in the second leg stretching motion.

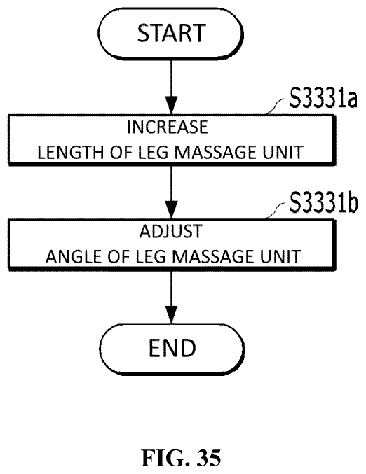

FIG. 35 is a diagram for describing a first leg stretching motion illustrated in FIG. 33. The controller 1400 may increase the length of the first leg massage unit 2000a while decreasing the length of the second leg massage unit 2000b through the leg length adjustment module 2500 (S3331a).

For example, the controller 1400 may tilt the leg massage unit 2000 in the up direction of the massage device 1000 as much as possible based on the maximum leg angle while the massage device 1000 is reclining.

The controller 1400 may increase the length of the first leg massage unit 2000a while decreasing the length of the second leg massage unit 2000b by a preset length while the leg massage unit 2000 is tilted as much as possible.

When reducing the length of the second leg massage unit 2000b, the controller 1400 may maximally move the second leg massage unit 2000b toward the up direction of the second leg massage unit 2000b while the second leg air cell 1730 is contracted to perform the second leg massage to reduce the length of the second leg massage unit 2000b to the shortest minimum length, but is not limited thereto. For example, the controller 1400 may reduce the length of the second leg massage unit 2000b based on the length of the other leg of the user, similar to the method of adjusting the length of the first leg massage unit 2000a, which will be described later.

When increasing the length of the first leg massage unit 2000a, the controller 1400 may expand (or while expanding) the first leg air cell 1710 and increase the length of the first leg massage unit 2000a based on the length of one leg of the user. As the method of expanding the first leg air cell 1710, a method of expanding leg air cells (e.g., first and second sub-calf air cells, first and second sub-foot air cells, etc.) may be applied in the S3010 operation of the piriformis muscle stretching mode.

For example, the controller 1400 may determine the adjustment length to increase the length of the first leg massage unit 2000a based on the length of one leg of the user. The controller 1400 may increase the length of the first leg massage unit 2000a by moving the first leg massage unit 2000a in the down direction of the first leg massage unit 2000a based on the adjustment length. In this case, the controller 1400 may move the first leg massage unit 2000a by the adjustment length or move the first leg massage unit 2000a to a position corresponding to the first adjustment length.

The above-described adjustment length determining method may be as follows. For example, the memory 1430 may store a leg length table including a plurality of leg lengths and an adjustment length matched to each leg length. The leg length table may be preset by various objects and stored in the memory 1430. The controller 1400 may extract an adjustment length that matches any one of a plurality of leg lengths in the leg length table based on the length of one leg of the user. In this case, the length of any one leg may be the same or similar to the length of one leg of the user. The controller 1400 may determine the extracted adjustment length as the adjustment length for adjusting the length of the first leg massage unit 2000a.

That is, the controller 1400 may expand only the first leg air cell 1710 to fix (or while fixing) one leg of the user to the first leg massage unit 2000a and adjust the length of the first leg massage unit 2000a to stretch one leg of the user.

In addition, when increasing the length of the first leg massage unit 2000a, the controller 1400 operates the first foot massage unit 2610 while the second foot massage unit 2630 is stopped to massage (or while massaging) the foot of one leg and increase the length of the first leg massage unit 2000a. In this case, the driving operation of the first foot massage unit 2610 may be linked to at least one of the expansion operation of the first leg air cell 1710 and the length increasing operation of the first leg massage unit 2000a. The driving operation of the first foot massage unit 2610 may end when the length increasing operation of the first leg massage unit 2000a is completed.

In other words, the controller 1400 may link at least one of the driving operation of the first foot massage unit 2610, the expansion operation of the first leg air cell 1710, and the length increasing operation of the first leg massage unit 2000a while the second foot massage unit 2630 is stopped and the second leg air cell 1730 is contracted to stretch one leg of the user.

The controller 1400 may adjust the angle of the first leg massage unit 2000a while maintaining the angle of the second leg massage unit 2000b through the leg angle adjustment module 1350 (S3331b).

For example, the controller 1400 may tilt the first leg massage unit 2000a in the down direction of the massage device 1000 while the length of the first leg massage unit 2000a increases and the first leg air cell 1710 is expanded to adjust the angle of the first leg massage unit 2000a.

When adjusting the angle of the first leg massage unit 2000a, the controller 1400 may adjust the angle of the first leg massage unit 2000a based on the length of one leg of the user.

For example, the controller 1400 may determine the adjustment angle to adjust the angle of the first leg massage unit 2000a based on the length of one leg of the user. The controller 1400 may adjust the angle of the first leg massage unit 2000a by tilting the first leg massage unit 2000a in the downward direction of the massage device 1000 based on the adjustment angle. In this case, the controller 1400 may tilt the first leg massage unit 2000a by the adjustment angle or tilt the first leg massage unit 2000a to the position corresponding to the adjustment angle.

The method for determining the adjustment angle described above may be similar to the method for determining the adjustment length. For example, the memory 1430 may store a leg angle table including a plurality of leg lengths and an adjustment length matched to each leg length. The leg angle table may be preset by various objects and stored in the memory 1430. The controller 1400 may extract an adjustment angle that matches any one of a plurality of leg lengths pre-stored in the memory 1430 based on the length of one leg of the user. In this case, the length of any one leg may be the same or similar to the length of one leg of the user. The controller 1400 may determine the extracted adjustment angle as the adjustment angle to adjust the angle of the first leg massage unit 2000a.

That is, the controller 1400 may stretch one leg of the user by adjusting the angle of the first leg massage unit 2000a while the length of the first leg massage unit 2000a increases and the first leg air cell 1710 is expanded.

As described above, the controller 1400 adjusts the angle of the first leg massage unit 2000a while maintaining the angle of the second leg massage unit 2000b, but is not limited thereto.

For example, the controller 1400 may perform at least one of the angle adjustment operation of the first leg massage unit 2000a, the reclining operation of the body massage unit 1100, the expansion operation of the body air cell 1600, and the buttock massage providing operation in conjunction with each other.

In the case of the reclining operation of the body massage unit 1100, the controller 1400 may adjust the angle of the first leg massage unit 2000a while reclining the body massage unit 1100 while maintaining the angle between the body massage unit 1100 and the second leg massage unit 2000b. For example, the controller 1400 may tilt the body massage unit 1100 toward the rear of the body massage unit 1100 as much as possible based on the maximum body angle.

In the case of the expansion operation of the body air cell 1600, the controller 1400 may expand the body air cell 1600 to move (or while moving) the user's body and then adjust the angle of the first leg massage unit 2000a. In this case, the controller 1400 may expand the body air cell 1600 after increasing (or while increasing) the length of the second leg massage unit 2000b.

As an example, the controller 1400 may expand at least one of the thigh air cells 1617 and 1637 and the perineum air cell 1650 to move (or while moving) the user's body in the up direction of the massage device 1000, and then adjust the angle of the first leg massage unit 2000a. At least one of the thigh air cells 1617 and 1637 and the perineum air cell 1650 may be expanded while the length of the first leg massage unit 2000a is increasing or the length of the first leg massage unit 2000a is increased.

As another example, the controller 1400 may expand at least one of the back air cells 1613 and 1633 and the pelvic air cells 1615 and 1635 to move (or while moving) the user's body to any one of both side portions of the massage device 1000 to adjust the angle of the first leg massage unit 2000a. At least one of the back air cells 1613 and 1633 and the pelvic air cells 1615 and 1635 may be expanded while the length of the first leg massage unit 2000a is increasing or the length of the first leg massage unit 2000a is increased.

For example, the at least one air cell may include at least one of the second back air cell 1633 and the second pelvic air cell 1635 corresponding to the second leg massage unit 2000b. In other words, the controller 1400 may expand at least one of the second back air cell 1633 and the second pelvic air cell 1635 to move the user's body to the side portion corresponding to the first leg massage unit 2000a among both side portions of the massage device 1000.

As another example, the controller 1400 may expand (or while expanding) at least one of the back air cells 1613 and 1633 and the pelvic air cells 1615 and 1635, and expand at least one of the thigh air cells 1617 and 1637 and the perineum air cell 1650 to move (or while moving) the user's body and then adjust the angle of the second leg massage unit 2000b.

In the case of the buttock massage providing operation, the controller 1400 may provide the buttock massage to the user's buttocks through the massage module 1110 before or after adjusting the angle of the first leg massage unit 2000a, or while adjusting the angle of the first leg massage unit 2000a.

Specifically, the controller 1400 may provide the buttock massage to the user by protruding the massage module 1110 in the front direction of the massage module 1110 and pressing the user's buttocks. In this case, the controller 1400 may provide the buttock massage to the user by protruding the massage module 1110 when the angle adjustment of the first leg massage unit 2000a is completed, but is not limited thereto. The buttock massage may include at least one of the acupressure massage, the kneading massage, the tapping massage, and the complex massage. The complex massage may be a massage that combines two or more of the acupressure massage, the kneading massage, and the tapping massage. At least some of the buttock massage operations included in the piriformis muscle stretching motion described above may be applied to the above-described buttock massage providing operation.

As described above, the controller 1400 may perform at least one of the angle adjustment operation of the first leg massage unit 2000a, the reclining operation of the body massage unit 1100, the expansion operation of the body air cell 1600, and the buttock massage providing operation in conjunction with each other, but is not limited thereto.

For example, the controller 1400 may perform the angle adjustment operation of the second leg massage unit 2000b in conjunction with the angle adjustment operation of the first leg massage unit.

Specifically, the controller 1400 may adjust the angle of the first leg massage unit 2000a while adjusting the angle of the second leg massage unit 2000b by tilting the second leg massage unit 2000b in at least one of the up and down directions of the massage device 1000. Preferably, since the second leg air cell 1710 of the second leg massage unit 2000b is contracted, the second leg massage unit 2000b may be tilted in the up direction of the massage device 1000.

In this way, the controller 1400 may quickly and accurately extend and relax the one leg (or the iliopsoas muscle of one leg) of the user, which is the leg to be stretched, and prevent unnecessary waste of power to expand the second leg air cell 1730 and reduce the occurrence of injuries to the user's body.

In addition, the controller 1400 may improve the stretching effect by further extending and relaxing the user's legs compared to the case where one leg of the user is not fixed to the first leg massage unit 2000a and the case where the body air cell 1600 is not expanded.

In addition, the controller 1400 may massage the foot of one leg of the user to make the user recognize that the one leg of the user is the leg to be stretched, thereby preventing the user injury and promoting (or inducing) the user's mental and physical stability.

In addition, the controller 1400 may extend and relax the one leg (or the iliopsoas muscle of one leg) of the user more quickly and accurately than when at least one of the reclining operation of the body massage unit 1100, the expansion operation of the body air cell 1600, and the buttock massage providing action is not performed.

In addition, the controller 1400 may extend and relax the one leg (or the iliopsoas muscle of one leg) of the user more quickly and accurately than when the angle adjustment operations of the first and second leg massage units are not performed in conjunction with each other.

In the above description, the technical idea of the present invention has been described along with the accompanying drawings, but this is an exemplary description of a preferred embodiment of the present invention and does not limit the present invention. In addition, it is clear that anyone skilled in the art of the present invention can make various modifications and imitations without departing from the scope of the technical idea of the present invention.

In addition, the configurations and functions of the massage device of the present specification may be expanded and applied to various (wearable) healthcare devices that may provide healthcare functions (e.g., massage/massage function, biological signal measurement function, etc.). Therefore, it goes without saying that the ‘massage device’ in this specification may be replaced with the ‘(wearable) healthcare device’.

1-19. (canceled)

20. A massage device massaging a body of a user and controlled by a controller, comprising:
a plurality of leg massage units that massage legs of the user,
wherein one leg massage unit of the plurality of leg massage units is controlled in at least one of a length and a angle by the controller, and the other leg massage unit of the plurality of leg massage units is controlled in at least one of a length and a angle differently from the one leg massage unit by the controller, and
an operation of controlling, by the controller, the plurality of leg massage units differently includes at least one of following operations:
fixing at least one of the length and the angle of the one leg massage unit and adjusting at least one of the length and the angle of the other massage unit;
when the massage device includes an air cell that massages or fixes the body of the user, controlling the one leg massage unit and the other leg massage unit in conjunction with the air cell; or
controlling the one leg massage unit and the other leg massage unit in an opposite manner to each other in at least one of the length and the angle.

21. The massage device, further comprising:
a leg connection frame,
wherein the one leg massage unit is rotatably connected to the leg connection frame, and
the other leg massage unit is connected to the leg connection frame separately from the one leg massage unit and is individually rotatably provided with the one leg massage unit.

22. The massage device of claim 21, further comprising:
a center support bracket that is provided on the leg connection frame and supports inner sides of the one leg massage unit and the leg massage unit; and
a side support bracket that is provided on the leg connection frame and supports outer sides of the one leg massage unit and the other leg massage unit.

23. The massage device of claim 22, wherein the one leg massage unit includes a first center bushing that is coupled to the center support bracket and a first side bushing that is coupled to the side support bracket, and
the other leg massage unit includes a second center bushing that is coupled to the center support bracket and a second side bushing that is coupled to the side support bracket.

24. The massage device of claim 23, further comprising:
a shaft that is coupled to one side of the body massage unit and connected to the leg massage unit,
wherein the shaft includes:
a first shaft that is coupled to the one leg massage unit; and
a second shaft that is coupled to the other leg massage unit.

25. The massage device of claim 24, wherein the first center bushing is coupled to one side of the first shaft, and the first side bushing is coupled to the other side of the first shaft, and
the second center bushing is coupled to one side of the second shaft, and the second side bushing is coupled to the other side of the second shaft.

26. The massage device of claim 23, wherein the center support bracket includes:
a center fixing member that is connected to the leg connection frame and provided with a center bushing receiving groove; and
a center rotation member that is rotatably provided on the center fixing member and provided with a center bushing fixing groove at a portion corresponding to the center bushing receiving groove, and
the side support bracket includes:
a side fixing member that is connected to the leg connection frame and provided with a side bushing receiving groove; and
a side rotation member that is rotatably provided on the side fixing member and provided with a side bushing fixing groove at a portion corresponding to the side bushing receiving groove.

27. The massage device of claim 23, wherein a first center support rib for preventing separation from the center support bracket is provided to protrude outward in a radial direction on an outer peripheral surface of the first center bushing,
a first side support rib for preventing separation from the side support bracket is provided to protrude outward in a radial direction on an outer peripheral surface of the first side bushing,
a second center support rib for preventing the separation from the center support bracket is provided to protrude outward in a radial direction on an outer peripheral surface of the second center bushing, and
a second side support rib for preventing the separation from the side support bracket is provided to protrude outward in a radial direction on an outer peripheral surface of the second side bushing.

28. The massage device of claim 20, wherein, when the controller adjusts at least one of the length and the angle of the other leg massage unit, the controller increases the length of the other leg massage unit and then tilts the other leg massage unit upward or downward to adjust the angle of the other leg massage unit.

29. The massage device of claim 20, wherein, when the controller fixes at least one of the length and the angle of the one leg massage unit and adjusts at least one of the length and the angle of the other leg massage unit differently from the one leg massage unit, the controller performs at least one of following operations:
adjusting the length of the other leg massage unit while fixing the length of the one leg massage unit;
adjusting the angle of the other leg massage unit while fixing the angle of the one leg massage unit;
adjusting the angle of the other leg massage unit while fixing the length of the one leg massage unit; or
adjusting the length of the other leg massage unit while fixing the angle of the one leg massage unit.

30. The massage device of claim 20, wherein the body massage unit includes a first body air cell that is disposed on one side of the body massage unit to massage one side of the body of the user, and a second body air cell that is disposed on the other side of the body massage unit to massage the other side of the body of the user.

31. The massage device of claim 30, wherein, when the one leg massage unit and the other leg massage unit are controlled in conjunction with the air cells,
the controller controls at least one of the length and the angle of the one leg massage unit in conjunction with at least one of the first and second body air cells, and
controls at least one of the length and the angle of the other leg massage unit in conjunction with at least one of the first and second body air cells.

32. The massage device of claim 31, wherein the first body air cell includes at least one of:
a first back air cell that is disposed in an area corresponding to one side of a back of the user and massages the one side of the back;
a first pelvis air cell that is disposed in an area corresponding to one side of a pelvis of the user and massages the one side of the pelvis;
a first shoulder air cell that is disposed in an area corresponding to one side of a shoulder of the user and massages the one side of the shoulder; or
a first thigh air cell that is disposed in an area corresponding to a thigh of one leg and massages the thigh of the one leg; and
the second body air cell includes at least one of:
a second back air cell that is disposed in an area corresponding to the other side of the back of the user and massages the other side of the back;
a second pelvis air cell that is disposed in an area corresponding to the other side of the pelvis of the user and massages the other side of the pelvis;
a second shoulder air cell that is disposed in an area corresponding to the other side of the shoulder of the user and massages the other side of the shoulder; or
a second thigh air cell that is disposed in an area corresponding to a thigh of the other leg of the user and massages the thigh of the other leg.

33. The massage device of claim 20, wherein, when the controller controls the one leg massage unit and the other leg massage unit in an opposite manner to each other in the length,
the controller decreases the length of the other leg massage unit while increasing the length of the one leg massage unit, or
increases the length of the other leg massage unit while decreasing the length of the one leg massage unit.

34. The massage device of claim 20, wherein, when the controller controls the one leg massage unit and the other leg massage unit in an opposite manner to each other in the angle,
the controller tilts the angle of the other leg massage unit downward while tilting the angle of the one leg massage unit upward, or
tilts the angle of the other leg massage unit upward while tilting the angle of the one leg massage unit downward.

35. The massage device of claim 20, wherein, when the controller controls the one leg massage unit and the other leg massage unit in an opposite manner to each other in at least one of the length and the angle,
the controller performs at least one of following operations of:
decreasing the length of the other leg massage unit and tilting the angle of the other leg massage unit upward while increasing the length of the one leg massage unit and tilting the angle of the one leg massage unit downward,
tilting the angle of the one leg massage unit upward while decreasing the length of the one leg massage unit and tilting the angle of the other leg massage unit downward while increasing the length of the other leg massage unit,
tilting the angle of the one leg massage unit upward while increasing the length of the one leg massage unit and tilting the angle of the other leg massage unit downward while decreasing the length of the other leg massage unit, or
tilting the angle of the one leg massage unit downward while decreasing the length of the one leg massage unit and tilting the angle of the other leg massage unit upward while increasing the length of the other leg massage unit.

36. The massage device of claim 20, wherein the controller adjusts the length of at least one of the one leg massage unit and the other leg massage unit while tilting the angle of at least one of the one leg massage unit and the other leg massage unit.

37. The massage device of claim 20, wherein the controller decreases the length of the one leg massage unit and tilts the angle of the one leg massage unit upward, relative to the other leg massage unit, or increases the length of the one leg massage unit and tilts the angle of the one leg massage unit downward, relative to the leg massage unit.

38. The massage device of claim 20, wherein, when the controller controls the one leg massage unit and the other leg massage unit in an opposite manner to each other in at least one of the length and the angle,
at least one air cell included in the one leg massage unit and the other leg massage unit is expanded to fix or massage the legs of the user received in the one leg massage unit and the other leg massage unit.

39. The massage device of claim 20, further comprising:
a leg length measuring sensor that measures the length of the legs of the user,
wherein, when the controller adjusts the angle of at least one of the one leg massage unit and the other leg massage unit, the controller determines an angle adjustment value of the one leg massage unit and the other leg massage unit based on the measured length of the legs of the user.
